(19) United States
(12) Patent Application Publication
(10) Pub. No.: US 2024/0130904 A1
(43) Pub. Date: Apr. 25, 2024

(54) MULTI-LAYERED ABSORBENT MATERIAL

(71) Applicant: Kimberly-Clark Worldwide, Inc., Neenah, WI (US)

(72) Inventors: Jian Qin, Appleton, WI (US); Francis P. Abuto, Johns Creek, GA (US); Sara L. Rosack, Greenville, WI (US); Stephen A. Marrano, Oshkosh, WI (US); Stephen M. Lindsay, Appleton, WI (US)

(21) Appl. No.: 18/278,090

(22) PCT Filed: Feb. 25, 2022
(86) PCT No.: PCT/US22/17859

§ 371 Date: Aug. 20, 2023

Related U.S. Application Data
(60) Provisional application No. 63/153,977, filed on Feb. 26, 2021.

(51) Int. Cl. A61F 13/539

(52) U.S. Cl. CPC A61F 13/539

(57) ABSTRACT

Absorbent materials described herein can include an intake layer and an absorbent layer. The absorbent material can include a saturation capacity greater than 125 grams, and a second intake time of less than 50 seconds and a wet thickness of less than 17 mm according to the Modified Fluid Intake Under Pressure Test as described herein. In some aspects, the intake layer and the absorbent layer can provide an integrated material including an interface between the intake layer and the absorbent layer. The interface can include at least some fibers of the intake layer mixed with at least some fibers of the absorbent layer.

### TECHNICAL FIELD

The present disclosure relates to multi-layered materials and apparatuses and methods for forming such materials. More specifically, the present disclosure relates to multi-layered absorbent materials.

### BACKGROUND OF THE DISCLOSURE

Personal care products, such as diapers, diaper pants, training pants, adult incontinence products, and feminine care products, can include absorbent structures that are intended to provide various functional characteristics. For example, absorbent structures in such products are intended to intake body exudates sufficiently quickly, distribute such exudates to an absorbent core or body that is capable of storing an adequate volume of exudates, and prevent such stored exudates in the absorbent core from exiting the absorbent core and transferring to other layers of the product and/or against the user's skin or clothing. Personal care products must also be considerate of other user perceived benefits such as comfort and discreteness, which can be impacted by absorbent structure properties such as thickness (wet thickness and/or dry thickness), stiffness, and weight.

Producing a multilayer absorbent material with satisfactory properties in each of these categories proves difficult because while designing an absorbent structure to enhance one property can negatively affect other properties. For example, the storage capacity (saturation capacity) of an absorbent structure can be increased by adding more absorbent fibers or superabsorbent material into an absorbent layer of an absorbent structure, however, such addition of material can increase the thickness (dry and/or wet) of the absorbent material. Further, modifying the absorbent structure to improve the intake properties of the absorbent structure can negatively impact the rewet properties of the absorbent structure.

Thus, there exists a need to develop multi-layer absorbent materials that can provide different functionalities between various layers and that provide necessary intake functionality and saturation capacity, yet is able to do so while still remaining sufficiently thin.

### SUMMARY OF THE DISCLOSURE

In one embodiment, an absorbent material is provided. The absorbent material can include an intake layer and an absorbent layer. The absorbent material can include a saturation capacity greater than 125 grams, and a second intake time of less than 50 seconds and a wet thickness of less than 17 mm according to the Modified Fluid Intake Under Pressure Test as described herein.

In another embodiment, another absorbent material is provided. The absorbent material can include an intake layer and an absorbent layer. The absorbent material can include a dry thickness of less than 8.0 mm, a wet thickness of less than 12.5 mm, and a rewet less than or equal to 0.14 grams according to the Modified Fluid Intake Under Pressure Test as described herein.

In still another embodiment, an absorbent material is provided. The absorbent material can include an intake layer and an absorbent layer. The absorbent material can include a saturation capacity greater than 125 grams, and a rewet of less than or equal to 0.14 grams and a wet thickness of less than 17 mm according to the Modified Fluid Intake Under Pressure Test as described herein.

In yet another embodiment, an absorbent material can include an intake layer. The intake layer can include synthetic fibers and binder fibers. The intake layer can include a basis weight less than 50 gsm. The absorbent material can also include an absorbent layer. The absorbent layer can include superabsorbent material, cellulosic fibers, and binder fibers. The binder fibers can provide less than 20% of the absorbent layer (by total weight of the absorbent layer). The intake layer and the absorbent layer can provide an integrated material including an interface between the intake layer and the absorbent layer. The interface can include at least some fibers of the intake layer with at least some fibers of the absorbent layer.

### BRIEF DESCRIPTION OF THE DRAWINGS

A full and enabling disclosure thereof, directed to one of ordinary skill in the art, is set forth more particularly in the remainder of the specification, which makes reference to the appended figures in which:

FIG. 1A is a side plan view of an exemplary multi-layer absorbent material including three layers according to one embodiment of the present disclosure.

FIG. 1B is a side plan view of an exemplary multi-layer absorbent material including two layers according to another embodiment of the present disclosure.

FIG. 1C is a side plan view of another exemplary multi-layer absorbent material including two layers according to another embodiment of the present disclosure.

FIG. 2 is a process schematic of an exemplary apparatus and associated method for forming a multi-layer absorbent material.

FIG. 3 is a detailed view of the headbox, headbox inputs, and resultant slurry from the headbox of FIG. 2.

FIG. 4 is a side plan view of an alternative apparatus and associated method that can be used for forming a multi-layer absorbent material.

FIG. 5 is a perspective view of exemplary equipment for performing the Fluid Intake Under Pressure (FIUP) Test described herein with the cover being opened.

FIG. 6 is a perspective view of the exemplary equipment of FIG. 5 with the cover being closed.

FIG. 7A is a perspective view of exemplary equipment of the Horizontal Compression Test described herein.

FIG. 7B is a perspective view of other exemplary equipment of the Horizontal Compression Test described herein.

FIG. 8 is a front plan view of exemplary equipment for performing the Pad Shake Test described herein.

Repeat use of reference characters in the present specification and drawings is intended to represent the same or analogous features or elements of the disclosure.

### DETAILED DESCRIPTION OF THE DISCLOSURE

Each example is provided by way of explanation and is not meant as a limitation. For example, features illustrated or described as part of one embodiment or figure can be used on another embodiment or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. As used herein, the terminology of “first,” “second,” “third”, etc. does not designate a specified order, but is used as a means to differentiate between different occurrences when referring to various features in the present disclosure. Many modifications and variations of the present disclosure can be made without departing from the spirit and scope thereof. Therefore, the exemplary embodiments described herein should not be used to limit the scope of the invention.

### Definitions

As used herein, the term “foam formed product” means a product formed from a suspension including a mixture of a solid, a liquid, and dispersed gas bubbles.

As used herein, the term “foam forming process” means a process for manufacturing a product involving a suspension including a mixture of a solid, a liquid, and dispersed gas bubbles.

As used herein, the term “foaming fluid” means any one or more known fluids compatible with the other components in the foam forming process. Suitable foaming fluids include, but are not limited to, water.

As used herein, the term “foam half life” means the time elapsed until the half of the initial frothed foam mass reverts to liquid water.

As used herein, the term “layer” refers to a structure that provides an area of a substrate in a z-direction of the substrate that is comprised of similar components and structure.

As used herein, the term “nonwoven web” means a web having a structure of individual fibers or threads which are interlaid, but not in an identifiable manner as in a knitted web.

As used herein, unless expressly indicated otherwise, when used in relation to material compositions the terms “percent”, “%”, “weight percent”, or “percent by weight” each refer to the quantity by weight of a component as a percentage of the total except as whether expressly noted otherwise.

The term “personal care absorbent article” refers herein to an article intended and/or adapted to be placed against or in proximity to the body (i.e., contiguous with the body) of the wearer to absorb and contain various liquid, solid, and semi-solid exudates discharged from the body. Examples include, but are not limited to, diapers, diaper pants, training pants, youth pants, swim pants, feminine hygiene products, including, but not limited to, menstrual pads or pants, incontinence products (e.g., bed mats), medical garments, surgical pads and bandages, and so forth.

The term “ply” refers to a discrete layer within a multi-layered product wherein individual plies may be arranged in juxtaposition to each other.

The term “plied” or “bonded” or “coupled” refers herein to the joining, adhering, connecting, attaching, or the like, of two elements. Two elements will be considered plied, bonded or coupled together when they are joined, adhered, connected, attached, or the like, directly to one another or indirectly to one another, such as when each is directly bonded to intermediate elements. The plying, bonding or coupling of one element to another can occur via continuous or intermittent bonds.

The term “superabsorbent material” as used herein refers to water-swellable, water-insoluble organic or inorganic materials including superabsorbent polymers and superabsorbent polymer compositions capable, under the most favorable conditions, of absorbing at least about 10 times their weight, or at least about 15 times their weight, or at least about 25 times their weight in an aqueous solution containing 0.9 weight percent sodium chloride.

### Multi-Layer Absorbent Material

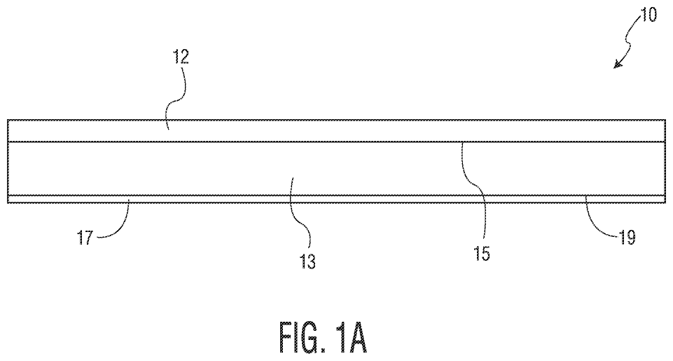

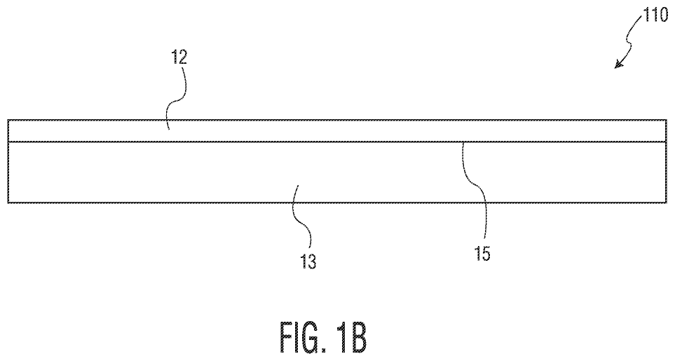

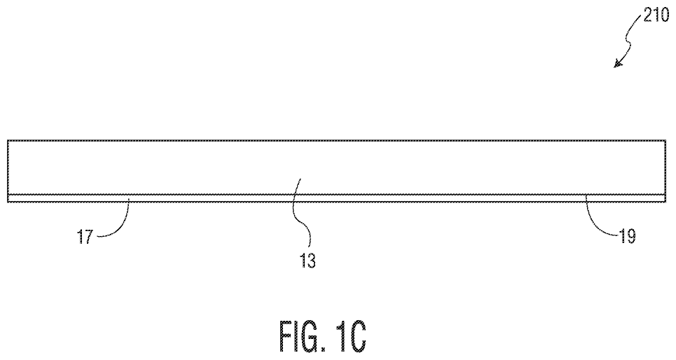

The present disclosure is directed to multi-layer absorbent materials, such as the absorbent materials 10, 110, 210 illustrated in FIGS. 1A-1C. These absorbent materials 10, 110, 210 can also be referred to as absorbent substrates 10, 110, 210 herein. In some embodiments, multi-layer absorbent materials 10, 110, 210 can include at least two layers. In some embodiments, multi-layer absorbent materials 110, 210 can include two layers (such as illustrate in FIGS. 1B, 1C), while in other embodiments, multi-layer absorbent materials 10 can include three or more layers (such as illustrated in FIG. 1A). The absorbent materials 10, 110, 210 of the present disclosure can include natural fibers and/or synthetic fibers. In some embodiments, the multi-layer absorbent materials 10, 110, 210 can include additional components, additives, and/or binders in one or more layers of the absorbent material 10, 110, 210 as further described herein.

In some preferred embodiments, the absorbent material 10, 110 can include an intake layer 12 and an absorbent layer 13. The intake layer 12 is generally configured to intake fluids, such as body exudates, and can include natural and/or synthetic fibers, as described further below. The absorbent layer 13 is generally configured to absorb such fluids and includes absorbent material, including absorbent fibers and/or absorbent components, such as, superabsorbent material.

In some preferred embodiments, the absorbent material 10, 210 can include a containment layer 17, as illustrated in the embodiment of the absorbent materials of FIGS. 1A and 1C. The containment layer 17 is generally configured to contain the absorbent layer 13, and in particular, particulates or fibers that may be included in the absorbent layer 13. As depicted in FIG. 1A, in embodiments including a three layered absorbent material 10, the absorbent layer 13 can be disposed between the intake layer 12 and the containment layer 17.

In preferred embodiments discussed herein, the multi-layer absorbent material 10, 210 can be configured to provide an integrated material such that the absorbent material 10 includes an interface 19 between the absorbent layer 13 and the containment layer 17 that includes at least some fibers of the containment layer 17 mixed with at least some of the fibers or particulates of the absorbent layer 13. The interface 19 can provide the benefit of having some fiber distribution between each of the absorbent layer 13 and the containment layer 17 that can provide enhanced stabilization properties between the two layers 13, 17.

In some embodiments, the absorbent material 10, 110 can also include an interface 15 between the intake layer 12 and the absorbent layer 13 that includes at least some fibers of the intake layer 12 mixed with at least some of the fibers of the absorbent layer 13. The interface 15 can provide the benefit of having some fiber distribution between each of the intake layer 12 and the absorbent layer 13 that can provide intake benefits, as well as some stabilization properties between the two layers. Additionally, in preferred embodiments including binder fibers in at least one of the intake layer 12 and the absorbent layer 13, the interface 15 can also provide the benefit of enhanced stabilization between the layers 12, 13.

The absorbent substrate 10, 210 can be formed with various properties in the containment layer 17. For example, the absorbent substrate 10 can be formed such that containment layer 17 includes a thickness of between about 0.10 mm to about 1.00 mm, and in some embodiments from about 0.15 mm to about 0.80 mm, and in other embodiments from about 0.20 mm to about 0.4 mm. The basis weight of the containment layer 17 can include a basis weight from about 5 gsm to 50 gsm, or in some embodiments from about 10 gsm to about 40 gsm, or about 10 gsm to about 25 gsm. As will be discussed further below, the containment layer 17 can be configured to protect the absorbent substrate 10, especially one that includes particulate components 44, from dewatering and/or drying conditions on the substrate 10, 210 to prevent such components 44 from becoming lodged in or drawn through forming surfaces 94 during wet processes, such as foam forming.

In some embodiments, the containment layer 17 can include cellulosic fibers, as such fibers provide benefits of wicking and low basis weight containment. Of course, in some embodiments, the containment layer 17 can include other fiber types described herein in addition to or in place of cellulosic fibers. For example, the containment layer 17 can include bicomponent fibers to be used as a binder material to provide enhanced integrity to the containment layer 17 and/or the absorbent material 10, 110, 210. In some embodiments, the containment layer 17 can include three-dimensional synthetic fibers, such as crimped synthetic fibers, that can provide larger pore sizes for increased bulk and improved intake. In some embodiments, the containment layer 17 may include some components 44, such as superabsorbent material (SAM), that may migrate from the foam-forming processes as described herein when forming the absorbent layer 13.

In some embodiments, the absorbent layer 13 can include at least some fibers, which can include cellulosic fibers. In some embodiments, the absorbent layer 13 can also include binders, such as binder fibers. In preferred embodiments, the absorbent layer 13 can include superabsorbent material as a component 44, which is often provided in particulate form. The absorbent layer 13 can be modified to include various basis weights and thicknesses depending on the intended product application for the absorbent substrate 10, 110, 210.

In some embodiments, the intake layer 12 can include synthetic fibers. In some preferred embodiments, the intake layer 12 can also include a binder, such as binder fibers, in addition to the synthetic fibers.

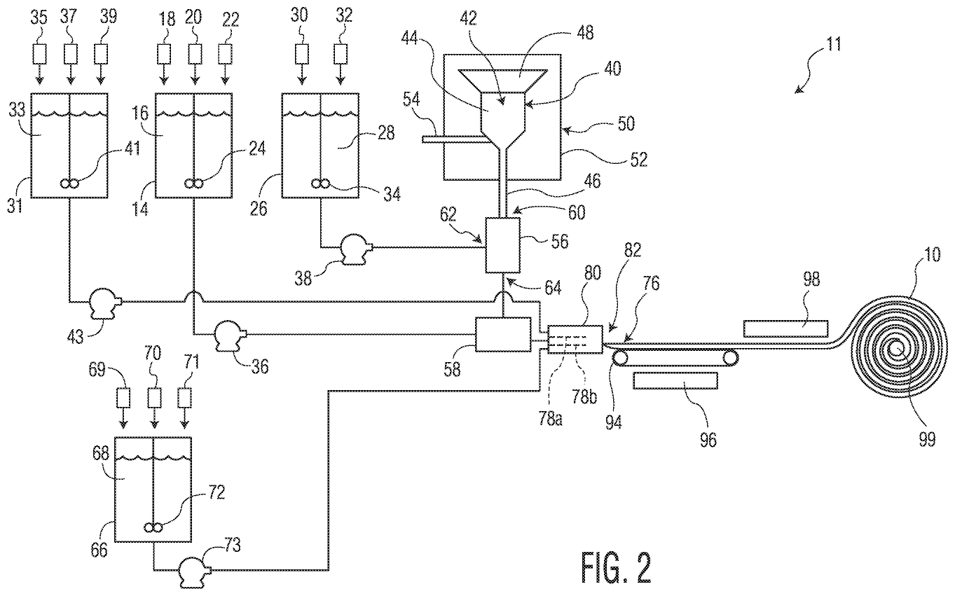

Preferred absorbent materials 10, 210 of the present disclosure including such an interface 15 between the absorbent layer 13 and the containment layer 17 can be formed through a foam forming process. Exemplary foam forming apparatuses and methods 11, 111 are described herein with respect to FIGS. 2-4.

It is to be noted that reference in this disclosure to absorbent materials 10, can refer to absorbent materials 110, 210, and vice versa, unless expressly noted otherwise.

Fibers

A wide variety of cellulosic fibers are believed suitable for use in the absorbent materials 10, 110, 210 described herein. In some embodiments, cellulosic fibers can be utilized in the absorbent layer 13, the containment layer 17, and/or the intake layer 12. In some embodiments, the fibers utilized can be conventional papermaking fibers such as wood pulp fibers formed by a variety of pulping processes, such as kraft pulp, sulfite pulp, bleached chemithermomechanical pulp (BCTMP), chemithermomechanical pulp (CTMP), pressure/pressure thermomechanical pulp (PTMP), thermomechanical pulp (TMP), thermomechanical chemical pulp (TMCP), and so forth. By way of example only, fibers and methods of making wood pulp fibers are disclosed in U.S. Pat. No. 4,793,898 to Laamanen et al.; U.S. Pat. No. 4,594,130 to Chang et al.; U.S. Pat. No. 3,585,104 to Kleinhart; U.S. Pat. No. 5,595,628 to Gordon et al.; U.S. Pat. No. 5,522,967 to Shet; and so forth. Further, the fibers may be any high-average fiber length wood pulp, low-average fiber length wood pulp, or mixtures of the same. Examples of suitable high-average length pulp fibers include softwood fibers, such as, but not limited to, northern softwood, southern softwood, redwood, red cedar, hemlock, pine (e.g., southern pines), spruce (e.g., black spruce), and the like. Examples of suitable low-average length pulp fibers include hardwood fibers, such as, but not limited to, eucalyptus, maple, birch, aspen, and the like.

Moreover, if desired, secondary fibers obtained from recycled materials may be used, such as fiber pulp from sources such as, for example, newsprint, reclaimed paperboard, and office waste. In some embodiments, refined fibers can be such that the total amount of virgin and/or high average fiber length wood fibers, such as softwood fibers, may be reduced.

Regardless of the origin of the wood pulp fiber, the wood pulp fibers preferably have an average fiber length greater than about 0.2 mm and less than about 3 mm, such as from about 0.35 mm and about 2.5 mm, or between about 0.5 mm to about 2.5 mm or even between about 0.7 mm and about 2.0 mm.

In addition, other cellulosic fibers that can be used in the present disclosure includes nonwoody fibers. As used herein, the term “non-wood fiber” generally refers to cellulosic fibers derived from non-woody monocotyledonous or dicotyledonous plant stems. Non-limiting examples of dicotyledonous plants that may be used to yield non-wood fiber include kenaf, jute, flax, ramie and hemp. Non-limiting examples of monocotyledonous plants that may be used to yield non-wood fiber include cereal straws (wheat, rye, barley, oat, etc.), stalks (corn, cotton, sorghum, Hesperaloe funifera, etc.), canes (bamboo, sisal, bagasse, etc.) and grasses (miscanthus. esparto, lemon, sabai, switchgrass, etc). In still other certain instances non-wood fiber may be derived from aquatic plants such as water hyacinth, microalgae such as Spirulina, and macroalgae seaweeds such as red or brown algae.

Still further, other cellulosic fibers for making substrates herein can include synthetic cellulose fiber types formed by spinning, including rayon in all its varieties, and other fibers derived from viscose or chemically-modified cellulose such as, for example, those available under the trade names LYOCELL and TENCEL.

Crosslinked cellulosic fibers, such as CMC 535, can also be used in forming materials 10, 110, 210 described herein. Crosslinked cellulosic fibers can provide increased bulk and resiliency, as well as improved softness.

In some embodiments, the non-woody and synthetic cellulosic fibers can have fiber length greater than about 0.2 mm including, for example, having an average fiber size between about 0.5 mm and about 50 mm or between about 0.75 and about 30 mm or even between about 1 mm and about 25 mm. Generally speaking, when fibers of relatively larger average length are being used, it may often be advantageous to modify the amount and type of foaming surfactant. For example, in some embodiments, if fibers of relatively larger average length are being used, it may be beneficial to utilize relatively higher amounts of foaming surfactant in order to help achieve a foam with the required foam half life.

Additional fibers that may be utilized in the present disclosure include synthetic fibers that are non-absorbent. In preferred embodiments, the intake layer 12 of the absorbent material 10, 110 can include synthetic fibers that are non-absorbent. In some embodiments, the absorbent layer 13 and/or the containment layer 17 can include synthetic fibers that are non-absorbent. As will be discussed below, in foam forming processes that may be advantageous for forming the multi-layer absorbent material 10, 110, 210 typically the forming fluid will comprise water. Synthetic non-absorbent fibers can have a bending stiffness that is substantially unimpacted by the presence of forming fluid. By way of non-limiting example, water-resistant fibers include fibers such as polymeric fibers comprising polyolefin, polyester (PET), polyamide, polylactic acid, or other fiber forming polymers. Polyolefin fibers, such as polyethylene (PE) and polypropylene (PP), are particularly well suited for use in the present disclosure. In some embodiments, non-absorbent fibers can be recycled fibers, compostable fibers, and/or marine degradable fibers. In addition, highly cross-linked cellulosic fibers having no-significant absorbent properties can also be used herein. In this regard, due to its very low levels of absorbency to water, water resistant fibers do not experience a significant change in bending stiffness upon contacting an aqueous fluid and therefore are capable of maintain an open composite structure upon wetting. The fiber diameter of a fiber can contribute to enhanced bending stiffness. For example, a PET fiber has a higher bending stiffness than a polyolefin fiber whether in dry or wet states. The higher the fiber denier, the higher the bending stiffness a fiber exhibits. Water resistant fibers desirably have a water retention value (WRV) less than about 1 and still more desirably between about 0 and about 0.5. In certain aspects, it is desirable that the fibers, or at least a portion thereof, include non-absorbent fibers.

The synthetic and/or water resistant fibers can have fiber length greater than about 0.2 mm including, for example, having an average fiber size between about 0.5 mm and about 50 mm or between about 0.75 and about 30 mm or even between about 1 mm and about 25 mm.

In some embodiments, the synthetic and/or water resistant fibers can have a crimped structure to enhance bulk generation capability of the foam formed fibrous substrate. For example, a PET crimped staple fiber may be able to generate a higher caliper (or result in a low sheet density) in comparison to a PET straight staple fiber with the same fiber diameter and fiber length.

Binder Materials

In preferred embodiments, binder materials can also form part of the absorbent material 10, 110, 210. Binder materials that may be used in the present disclosure can include, but are not limited to, thermoplastic binder fibers, such as PET/PE bicomponent binder fiber, and water-compatible adhesives such as, for example, latexes. In some embodiments, binder materials as used herein can be in powder form, for example, such as thermoplastic PE powder. Importantly, the binder can comprise one that is water insoluble on the dried substrate. In certain embodiments, latexes used in the present disclosure can be cationic or anionic to facilitate application to and adherence to cellulosic fibers that can be used herein. For instance, latexes believed suitable for use include, but are not limited to, anionic styrene-butadiene copolymers, polyvinyl acetate homopolymers, vinyl-acetate ethylene copolymers, vinyl-acetate acrylic copolymers, ethylene-vinyl chloride copolymers, ethylene-vinyl chloride-vinyl acetate terpolymers, acrylic polyvinyl chloride polymers, acrylic polymers, nitrile polymers, as well as other suitable anionic latex polymers known in the art. Examples of such latexes are described in U.S. Pat. No. 4,785,030 to Hager, U.S. Pat. No. 6,462,159 to Hamada, U.S. Pat. No. 6,752,905 to Chuang et al. and so forth. Examples of suitable thermoplastic binder fibers include, but are not limited to, monocomponent and multi-component fibers having at least one relatively low melting thermoplastic polymer such as polyethylene. In certain embodiments, polyethylene/polypropylene sheath/core staple fibers can be used. Binder fibers may have lengths in line with those described herein above in relation to the synthetic cellulosic fibers.

Additional Components

In some embodiments, the absorbent material 10, 110, 210 can include one or more additive components. For example, in preferred embodiments the absorbent material 10, 110, 210 can include superabsorbent material (SAM) in the absorbent layer 13 of the material 10, 110, 210. SAM is commonly provided in a particulate form and, in certain aspects, can comprise polymers of unsaturated carboxylic acids or derivatives thereof. In some forms, however, SAM can be configured in fiber form. These polymers are often rendered water insoluble, but water swellable, by crosslinking the polymer with a di- or polyfunctional internal crosslinking agent. These internally cross-linked polymers are at least partially neutralized and commonly contain pendant anionic carboxyl groups on the polymer backbone that enable the polymer to absorb aqueous fluids, such as body fluids. Typically, the SAM particles are subjected to a post-treatment to crosslink the pendant anionic carboxyl groups on the surface of the particle. SAMs are manufactured by known polymerization techniques, desirably by polymerization in aqueous solution by gel polymerization. The products of this polymerization process are aqueous polymer gels, i.e., SAM hydrogels that are reduced in size to small particles by mechanical forces, then dried using drying procedures and apparatus known in the art. The drying process is followed by pulverization of the resulting SAM particles to the desired particle size. Examples of superabsorbent materials include, but are not limited to, those described in U.S. Pat. No. 7,396,584 Azad et al., U.S. Pat. No. 7,935,860 Dodge et al., US2005/5245393 to Azad et al., US2014/09606 to Bergam et al., WO2008/027488 to Chang et al. and so forth.

In some embodiments involving SAM, the SAM may be treated by a water-soluble protective coating having a rate of dissolution selected such that the component is not substantially exposed to the aqueous liquid carrier until the highly-expanded foam has been formed and drying operations initiated that can remove the coating. Alternatively, in order to prevent or limit premature expansion during processing, the SAM may be introduced into the process at low temperatures.

In some embodiments incorporating SAM, the SAM can comprise between about 0% and about 40% of the foam (by weight). In certain embodiments, SAM can comprise between about 1% and about 30% of the foam (by weight) or even between about 10% and about 30% of the foam (by weight).

It has been surprisingly discovered that foam-forming methods as described herein are capable of forming absorbent substrates 10, 110, 210 with high percentages of SAM in the absorbent layer 13, such as greater than 80% of the absorbent layer 13 by total weight of the absorbent layer 13 (as measured by the Sulfated Ash Test Method described herein). In some embodiments, absorbent substrates 10, 110, 210, can include SAM providing from greater than 80% up to even 100% of the absorbent layer 13 by total weight of the absorbent layer 13. In some embodiments, SAM can provide greater than 85%, greater than 90%, greater than 95%, and even greater than 97% of the absorbent layer 13 by total weight of the absorbent layer 13. Previously, it was believed that foam-forming methods were not capable of providing such a high percentage of SAM in the absorbent layer 13 and still retain adequate integrity of the absorbent layer 13 and provide proper SAM retention in the absorbent layer 13.

In preferred embodiments, high percentage SAM absorbent layers 13, such as absorbent layers 13 with greater than 80% SAM, can benefit from having fibers in the absorbent layer 13 with a fiber length greater than about 0.8 mm, or greater than about 1.0 mm, or more preferably greater than about 1.25 mm, or even more preferably greater than about 1.50 mm, as provided by a length weighted average. One beneficial fiber having a length of this length are NBSK fibers, Northern Bleached Softwood Kraft, a commercial northern softwood pulp fiber, which often have fiber lengths of about 1.9 mm to about 2.1 mm. In some embodiments, the absorbent layer 13 includes absorbent fibers (NBSK being one exemplary type of absorbent fibers). Additionally, some embodiments of absorbent materials 10, 110, 210 described herein can include an absorbent layer 13 with synthetic material fibers having a length capable of providing additional stability to the absorbent layer 13. For example, some embodiments of the absorbent materials 10, 110, 210 can include an absorbent layer 13 with synthetic fibers having a length greater than about 4.0 mm, or more preferably greater than about 5.0 mm. Some preferable embodiments include an absorbent layer 13 with synthetic fibers of PET, having fiber lengths of about 6.0 mm.

In some embodiments of absorbent materials 10, 110, 210 described herein, the absorbent layer 13 can also include binder fibers. In some embodiments, the absorbent layer 13 can include a plurality of fibers that can include at least 20% by weight absorbent fibers and at least 20% by weight binder fibers (by total weight of the fibers in the absorbent layer 13). Binder fibers can provide additional integrity to the absorbent layer 13 of the absorbent substrate 10, 110, 210, and thus, the absorbent substrate 10, 110, 210 overall.

Other additional agents can include one or more wet strength additives that can be added to the foam or fluid supply 16, 28, 33, 68 in order to help improve the relative strength of the ultra-low density composite cellulosic material, in the foam forming. Such strength additives suitable for use with paper making fibers and the manufacture of paper tissue are known in the art. Temporary wet strength additives may be cationic, nonionic or anionic. Examples of such temporary wet strength additives include PAREZ™ 631 NC and PAREZ® 725 temporary wet strength resins that are cationic glyoxylated polyacrylamides available from Cytec Industries, located at West Paterson, N.J. These and similar resins are described in U.S. Pat. No. 3,556,932 to Coscia et al. and U.S. Pat. No. 3,556,933 to Williams et al. Additional examples of temporary wet strength additives include dialdehyde starches and other aldehyde containing polymers such as those described in U.S. Pat. No. 6,224,714 to Schroeder et al.; U.S. Pat. No. 6,274,667 to Shannon et al.; U.S. Pat. No. 6,287,418 to Schroeder et al.; and US6365667 to Shannon et al., and so forth.

Permanent wet strength agents comprising cationic oligomeric or polymeric resins may also be used in the present disclosure. Polyamide-polyamine-epichlorohydrin type resins such as KYMENE 557H sold by Solenis are the most widely used permanent wet-strength agents and are suitable for use in the present disclosure. Such materials have been described in the following U.S. Pat. No. 3,700,623 to Keim; U.S. Pat. No. 3,772,076 to Keim; U.S. Pat. No. 3,855,158 to Petrovich et al.; US3899388 to Petrovich et al.; U.S. Pat. No. 4,129,528 to Petrovich et al.; U.S. Pat. No. 4,147,586 to Petrovich et al.; U.S. Pat. No. 4,222,921 to van Eenam and so forth. Other cationic resins include polyethylenimine resins and aminoplast resins obtained by reaction of formaldehyde with melamine or urea. Permanent and temporary wet strength resins may be used together in the manufacture of composite cellulosic products of the present disclosure. Further, dry strength resins may also optionally be applied to the composite cellulosic webs of the present disclosure. Such materials may include, but are not limited to, modified starches and other polysaccharides such as cationic, amphoteric, and anionic starches and guar and locust bean gums, modified polyacrylamides, carboxymethylcellulose, sugars, polyvinyl alcohol, chitosan, and the like.

When a wet or dry strength additive is used, it is preferable to select such an additive to be compatible with the foam agent used for the foam process. For example, when a strength additive is a cationic resin, due to incompatibility between a cationic and an anionic substance, a cationic surfactant is preferably used as a foam agent, or vice versa. A non-ionic surfactant is usually compatible with any cationic and anionic strength additives.

If used, such wet and dry strength additives can comprise between about 0.01 and about 5% of the dry weight of cellulose fibers. In certain embodiments, the strength additives can comprise between about 0.05% and about 2% of the dry weight of cellulose fibers or even between about 0.1% and about 1% of the dry weight of cellulose fibers.

Still other additional components may be added to absorbent material 10, 110, 210. For materials 10, 110, 210 that are formed utilizing foam forming processes, other additional components should be reviewed as to ensure they do not significantly interfere with the formation of the foam, the hydrogen bonding as between the cellulosic fibers or other desired properties of the material 10, 110, 210. As examples, additional additives may include one or more pigments, opacifying agents, anti-microbial agents, pH modifiers, skin benefit agents, odor absorbing agents, fragrances, thermally expandable microspheres, foam particles (such as, pulverized foam particles), and so forth as desired to impart or improve one or more physical or aesthetic attributes. In certain embodiments the absorbent materials 10, 110, 210 may include skin benefit agents such as, for example, antioxidants, astringents, conditioners, emollients, deodorants, external analgesics, film formers, humectants, hydrotropes, pH modifiers, surface modifiers, skin protectants, and so forth.

### Foam Forming Method and Apparatus

Absorbent materials 10, 110, 210 as described herein can be preferably formed through a foam forming process. FIG. 2 provides a schematic of an exemplary apparatus 11 that can be used as part of a foam forming process to manufacture an absorbent material 10 that is a foam formed product. The apparatus 11 of FIG. 2 can include a first tank 14 configured for holding a first fluid supply 16. In some embodiments, the first fluid supply 16 can be a foam. The first fluid supply 16 can include a fluid provided by a supply of fluid 18. In some embodiments, the first fluid supply 16 can include a plurality fibers provided by a supply of fibers 20, and preferably includes at least some absorbent fibers. However, in other embodiments, the first fluid supply 16 can be free from a plurality of fibers altogether. The first fluid supply 16 can also include a surfactant provided by a supply of surfactant 22. In some embodiments, the first tank 14 can include a mixer 24, as will be discussed in more detail below. The mixer 24 can mix (e.g., agitate) the first fluid supply 16 to mix the fluid, fibers (if present), and surfactant with air, or some other gas, to create a foam. The mixer 24 can also mix the foam with fibers (if present) to create a foam suspension of fibers in which the foam holds and separates the fibers to facilitate a distribution of the fibers within the foam (e.g., as an artifact of the mixing process in the first tank 14). Uniform fiber distribution can promote desirable absorbent material 10 including, for example, strength and the visual appearance of quality.

The apparatus 11 can also include a second tank 26 configured for holding a second fluid supply 28. In some embodiments, the second fluid supply 28 can be a foam. The second fluid supply 28 can include a fluid provided by a supply of fluid 30 and a surfactant provided by a supply of surfactant 32. In some preferred embodiments, such as depicted in FIG. 2, the second fluid supply 28 is free from fibers. In other embodiments, the second fluid supply 28 can include a plurality of fibers in addition to or as an alternative to the fibers being present in the first fluid supply 16. In some embodiments, the second tank 26 can include a mixer 34. The mixer 34 can mix the second fluid supply 28 to mix the fluid and surfactant with air, or some other gas, to create a foam.

In some embodiments, the apparatus 11 can also include a third tank 31 configured for holding a third fluid supply 33. In some embodiments, the third fluid supply 33 can be a foam. The third fluid supply 33 can include a fluid provided by a supply of fluid 35 and a plurality of fibers provided by a supply of fibers 37, and preferably includes at least some synthetic fibers. The third fluid supply 33 can also include a surfactant provided by a supply of surfactant 39. In some embodiments, the third tank 31 can include a mixer 41. The mixer 41 can mix the third fluid supply 33 to mix the fluid and surfactant with air, or some other gas, to create a foam.

In some embodiments, the apparatus 11 can also include a fourth tank 66 configured for holding a fourth fluid supply 68. In some embodiments, the fourth fluid supply 68 can be a foam. The fourth fluid supply 68 can include a fluid provided by a supply of fluid 69 and a plurality of fibers provided by a supply of fibers 70. The fourth fluid supply 68 can also include a surfactant provided by a supply of surfactant 71. In some embodiments, the fourth tank 66 can include a mixer 72. The mixer 72 can mix the fourth fluid supply 68 to mix the fluid and surfactant with air, or some other gas, to create a foam.

In tanks 14, 26, 31, 66 the first fluid supply 16, the second fluid supply 28, the third fluid supply 33, and the fourth fluid supply 68, respectively, can be acted upon to form a foam. In some embodiments, the foaming fluid and other components are acted upon so as to form a porous foam having an air content greater than about 50% by volume and desirably an air content greater than about 60% by volume. In certain aspects, the highly-expanded foam is formed having an air content of between about 60% and about 95% and in further aspects between about 65% and about 85%. In certain embodiments, the foam may be acted upon to introduce air bubbles such that the ratio of expansion (volume of air to other components in the expanded stable foam) is greater than 1:1 and in certain embodiments the ratio of air:other components can be between about 1.1:1 and about 20:1 or between about 1.2:1 and about 15:1 or between about 1.5:1 and about 10:1 or even between about 2:1 and about 5:1.

The foam can be generated by one or more means known in the art. Examples of suitable methods include, without limitation, aggressive mechanical agitation such as by mixers 24, 34, 41, 72 injection of compressed air, and so forth. Mixing the components through the use of a high-shear, high-speed mixer is particularly well suited for use in the formation of the desired highly-porous foams. Various high-shear mixers are known in the art and believed suitable for use with the present disclosure. High-shear mixers typically employ a tank holding the foam precursor and/or one or more pipes through which the foam precursor is directed. The high-shear mixers may use a series of screens and/or rotors to work the precursor and cause aggressive mixing of the components and air. In a particular embodiment, the first tank 14, the second tank 26, the third tank 31, and/or the fourth tank 66 is provided having therein one or more rotors or impellors and associated stators. The rotors or impellors are rotated at high speeds in order to cause flow and shear. Air may, for example, be introduced into the tank at various positions or simply drawn in by the action of the mixers 24, 34, 41, 72. While the specific mixer design may influence the speeds necessary to achieve the desired mixing and shear, in certain embodiments suitable rotor speeds may be greater than about 500 rpm and, for example, be between about 1000 rpm and about 6000 rpm or between about 2000 rpm and about 4000 rpm. In other embodiments, suitable rotor speeds may be less than 500 rpm.

In addition, it is noted the foaming process can be accomplished in a single foam generation step or in sequential foam generation steps for the first tank 14, the second tank 26, the third tank 31, and/or the fourth tank 66. For example, in one embodiment, all of the components of the first fluid supply 16 in the first tank 14 (e.g., the supply of the fluid 18, fibers 20, and surfactant 22) may be mixed together to form a slurry from which a foam is formed. Alternatively, one or more of the individual components may be added to the foaming fluid, an initial mixture formed (e.g. a dispersion or foam), after which the remaining components may be added to the initially foamed slurry and then all of the components acted upon to form the final foam. In this regard, the fluid 18 and surfactant 22 may be initially mixed and acted upon to form an initial foam prior to the addition of any solids. Fibers, if desired, may then be added to the water/surfactant foam and then further acted upon to form the final foam. As a further alternative, the fluid 18 and fibers 20, such as a high density cellulose pulp sheet, may be aggressively mixed at a higher consistency to form an initial dispersion after which the foaming surfactant 22, additional water and other components, such as synthetic fibers, are added to form a second mixture which is then mixed and acted upon to form the foam.

The foam density of the foam forming the first fluid supply 16 in the first tank 14, the foam forming the second fluid supply 28 in the second tank 26, the third fluid supply 33 in the third tank 31, and/or the fourth fluid supply 68 in the fourth tank 66 can vary depending upon the particular application and various factors, such as the fiber stock used. In some implementations, for example, the foam density of the foam can be greater than about 100 g/L, such as greater than about 250 g/L, such as greater than about 300 g/L. The foam density is generally less than about 800 g/L, such as less than about 500 g/L, such as less than about 400 g/L, such as less than about 350 g/L. In some implementations, for example, a lower density foam is used having a foam density of generally less than about 350 g/L, such as less than about 340 g/L, such as less than about 330 g/L.

The apparatus 11 can also include a first pump 36, a second pump 38, third pump 43, and fourth pump 73. The first pump 36 can be in fluid communication with the first fluid supply 16 and can be configured for pumping the first fluid supply 16 to transfer the first fluid supply 16. The second pump 38 can be in fluid communication with the second fluid supply 28 and can be configured for pumping the second fluid supply 28 to transfer the second fluid supply 28. The third pump 43 can be in fluid communication with the third fluid supply 33 and can be configured for pumping the third fluid supply 33 to transfer the third fluid supply 33. The fourth pump 73 can be in fluid communication with the fourth fluid supply 68 and can be configured for pumping the fourth fluid supply 68 to transfer the fourth fluid supply 68. In some embodiments, the first pump 36, the second pump 38, the third pump 43, and/or the fourth pump 73 can be a progressive cavity pump or a centrifugal pump, however, it is contemplated that other suitable types of pumps can be used.

As depicted in FIG. 2, the apparatus 11 can also include a component feed system 40. The component feed system 40 can be used to deliver a supply of component 44, if one is desired for the absorbent material 10, by delivering the component 44 to one or more fluid supply 16, 28, 33, 68 or directly to the headbox 80. One exemplary component feed system 40 that can be used can include a component supply area 42 for receiving a supply of a component. The component feed system 40 can also include an outlet conduit 46. The component feed system 40 can also include a hopper 48. The hopper 48 can be coupled to the component supply area 42 and can be utilized for refiling the supply of the component 44 to the component supply area 42.

In some embodiments, the component feed system 40 can include a bulk solids pump. Some examples of bulk solids pumps that may be used herein can include systems that utilize screws/augers, belts, vibratory trays, rotating discs, or other known systems for handling and discharging the supply of the component 44. Other types of feeders can be used for the component feed system 40, such as, for example, an ingredient feeder, such as those manufactured by Christy Machine & Conveyor, Fremont, Ohio. The component feed system 40 can also be configured as a conveyor system in some embodiments.

In some embodiments, the component feed system 40 can also include a pressure control system 50. In some embodiments, the pressure control system 50 can include a housing 52. The housing 52 can form a pressurized seal volume around the component feed system 40. In other embodiments, the pressure control system 50 can be formed as an integral part to the structure component feed system 40 itself, such that a separate housing 52 surrounding the component feed system 40 may not be required. The pressure control system 50 can also include a bleed orifice 54 in some embodiments.

The supply of the component 44 can be in the form of a particulate and/or a fiber and/or a powder. In one embodiment as described herein, the supply of the component 44 can be superabsorbent material (SAM) in particulate form. In some embodiments, SAM can be in the form of a fiber. Of course, other types of components, as previously discussed, are also contemplated as being utilized in the apparatus 11 and methods for forming an absorbent material 10 as described herein. The component feed system 40 as described herein can be particularly beneficial for a supply of component 44 that is most suitably maintained in a dry environment with minimal of exposure to fluid or foam utilized in the apparatus 11 and methods described herein.

The apparatus 11 can also include a first mixing junction 56 and a second mixing junction 58. In preferred embodiments, the first mixing junction 56 can be an eductor (also commonly referred to as a jet pump). The first mixing junction 56 can be in fluid communication with the outlet conduit 46 of the component feed system 40 and in fluid communication with the second fluid supply 28. The first mixing junction 56 can include a first inlet 60 and a second inlet 62. The first inlet 60 can be in fluid communication with the supply of the component 44 via the outlet conduit 46. The second inlet 62 can be in fluid communication with the second fluid supply 28. The first mixing junction 56 can also include a discharge 64. In preferred embodiments, the first mixing junction 56 can be configured as a co-axial eductor with the axis of the first inlet 60 being co-axial with the axis of the outlet conduit 46 that provides the supply of the component 44. The first mixing junction 56 can also be configured such that the discharge axis of the discharge 64 is co-axial with the outlet axis of the outlet conduit 46. As such, the first mixing junction 56 can be configured such that the axis of the first inlet 60 can be co-axial with the axis of the discharge 64 of the first mixing junction 56. The second inlet 62 providing the second fluid supply 28 to the first mixing junction 56 can be set up to enter the first mixing junction 56 on a side of the first mixing junction 56.

When configured as an eductor, the first mixing junction 56 can mix the supply of the component 44 from the component feed system 40 with the second fluid supply 28. By transferring the second fluid supply 28 into the first mixing junction 56 at the second inlet 62 and through the first mixing junction 56, the second fluid supply 28 provides a motive pressure to the supply of the component 44. The motive pressure can create a vacuum on the supply of the component 44 and the component feed system 40 to help draw the supply of the component 44 to mix and be entrained in the second fluid supply 28. In some embodiments, the motive pressure can create a vacuum on the supply of the component 44 of less than 1.5 in Hg, however, in other embodiments, the motive pressure could create a vacuum on the supply of the component 44 of 5 in. Hg or more, or 10 in Hg or more.

The pressure control system 50 can help manage proper distribution and entrainment of the supply of the component 44 to the second fluid supply 28. For example, when the second fluid supply 28 creates a motive pressure on the component feed system 40, the vacuum pulling on the supply of the component 44 may cause additional air to be entrained in the second fluid supply 28. In some circumstances, entraining additional air in the second fluid supply 28 may be desired, however, in other circumstances, it may be desirable to control the gas content of the second fluid supply 28 while inputting the supply of the component 44 to the second fluid supply 28 at the first mixing junction 56. For example, in some circumstances where the second fluid supply 28 is a foam, the amount of gas content in the foam may be desired to be kept relatively fixed as the foam passes through the first mixing junction 56. Thus, the pressure control system 50 can control the pressure on the component feed system 40 to help counteract the motive pressure on the supply of the component 44 and the component feed system 40 created by the second fluid supply 28.

In some embodiments, the pressure control system 50 can include sealing off the component feed system 40. For example, as discussed above, the pressure control system 50 can include a housing 52 to provide a seal on the component feed system 40. Sealing the component feed system 40 can help to prevent additional air entrainment in the second fluid supply 28 when the supply of the component 44 is introduced into the second fluid supply 28 in the first mixing junction 56.

However, in some embodiments, it may be beneficial to also include additional capability to the pressure control system 50. For example, in some embodiments, the pressure control system 50 can include a bleed orifice 54. The bleed orifice 54 can be configured to bleed-in pressure, such as atmospheric air pressure, to provide additional pressure control of the component feed system 40. It has been discovered that by providing a bleed-in orifice 54 to provide some bleed-in of atmospheric air pressure to the component feed system 40, back-splashing of the second fluid supply 28 in the first mixing junction 56 can be reduced or eliminated. Reducing back-splashing of the second fluid supply 28 in the first mixing junction 56 can help prevent the component feed system 40 from becoming clogged or needing to be cleaned, especially where the component feed system 40 may be delivering a dry component, such as particulate SAM.

Additionally or alternatively, the pressure control system 50 can be configured to provide additional positive pressure to prevent back-filling of the component feed system 40 in some circumstances, such as if a downstream obstruction occurs in the apparatus 11 beyond the first mixing junction 56. In such a case of an obstruction creating an increased pressure, the second fluid supply 28 may have a desire to back-fill the component feed system 40. Back-filling of fluid into the component feed system 40 can be detrimental to processing, especially where the supply of the component 44 is a component best kept in dry conditions, such as SAM. A pressure control system 50 configured to be able to provide positive pressure to the component feed system 40 can help prevent such back-filling of the component feed system 40.

It is also contemplated that other additional aspects of a pressure control system 50 could be utilized to maintain the pressure to a suitable level for the component feed system 40, including, but not limited to, supplying vacuum to the component feed system 40 in addition to or alternative to the air bleed-in at the bleed orifice 54 and/or the positive pressure described above.

The first mixing junction 56 can also provide pressure control on the transfer of the second fluid supply 28 including the component 44 as it exits the discharge 64 of the first mixing junction 56 as compared to when the second fluid supply 28 enters the first mixing junction 56. The second fluid supply 28 can be transferred at a second fluid pressure prior to the first mixing junction 56. The second fluid supply 28 including the component from the supply of the component 44 can exit the discharge 64 of the first mixing junction 56 at a discharge pressure. The pressure difference between the second fluid pressure prior to the first mixing junction 56 and the discharge pressure can be controlled. In some embodiments, this pressure difference can be controlled by varying the flow rate of the second fluid supply 28 or through the positioning of the outlet conduit 46 in the first mixing junction 56. In some embodiments, it is preferable to control the pressure difference between the second fluid pressure prior to the first mixing junction 56 and the discharge pressure to be less than or equal to 5 pounds per square inch.

It is to be noted that while a single outlet conduit 46 of the component feed system 40 and a single first mixing junction 56 is illustrated in FIG. 2, it is contemplated that the outlet conduit 46 can be split into two or more conduits to feed two or more first mixing junctions 56 for mixing the supply of the component 44 with the second fluid supply 28. In such a configuration, the second fluid supply 28 can include as many conduits as there are first mixing junctions 56. By having more than one outlet conduit 46 and more than one first mixing junction 56 to mix the supply of the component 44 with the second fluid supply 28, a greater flow rate of the second fluid supply 28 including the component from the supply of the component 44 can be achieved.

Referring to FIG. 2, the apparatus 11 can include a second mixing junction 58 in some embodiments. The second mixing junction 58 can provide the functionality of mixing the second fluid supply 28 including the component from the supply of the component 44 with the first fluid supply 16. As the second fluid supply 28 including the component from the supply of the component 44 exits the discharge 64 of the first mixing junction 56 it can be transferred to the second mixing junction 58. The first fluid supply 16 can be delivered to the second mixing junction 58 by the first pump 36. The second mixing junction 58 can mix the first fluid supply 16 and any of its components (e.g., fluid 18, fibers 20, surfactant 22) with the second fluid supply 28 and any of its components (e.g., fluid 30, surfactant 32) and the component from the supply of the component 44 to deliver the mixture of the first fluid supply 16, the second fluid supply 28, and the component 44 to a headbox 80.

Alternatively, in some embodiments, a second mixing junction 58 can be omitted from the apparatus 11 and the second fluid supply 28 including the component from the supply of the component 44 can be delivered to headbox 80.

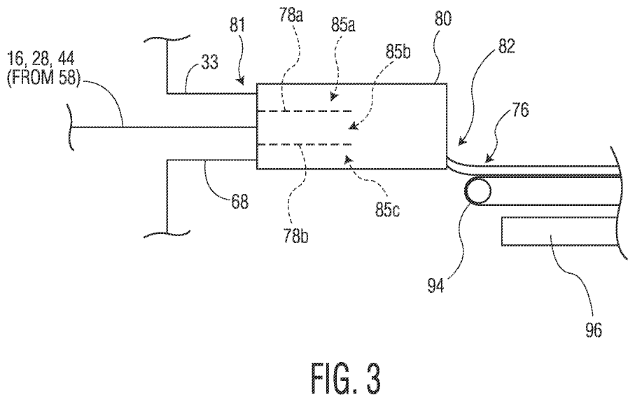

As illustrated in FIGS. 2 and 3, the headbox 80 can include one or more z-directional dividers 78a, 78b for separating different inputs to the headbox 80 in forming different layers of the absorbent material 10. The third fluid supply 33 and any of its components (e.g., fluid 35, fibers 37, surfactant 39) can be delivered to the inlet 81 of the headbox 80 via the third pump 43 and can be delivered above the first z-directional divider 78a in a first z-directional layer 85a of the headbox 80. The output of the second mixing junction 58 including the mixture of the first fluid supply 16 and any of its components (e.g., fluid 18, fibers 20, surfactant 22), the second fluid supply 28 and any of its components (e.g., fluid 30, surfactant 32), and the component 44 can be delivered to the inlet 81 of the headbox 80 below the first z-directional divider 78a and above the second z-directional divider 78b in a second z-directional layer 85b of the headbox 80. The fourth fluid supply 68 and any of its components (e.g., fluid 69, fibers 70, surfactant 71) can be delivered to the inlet 81 of the headbox 80 via the fourth pump 73 and can be delivered below the second z-directional divider 78b in a third z-directional layer 85c of the headbox 80. Such a configuration of two z-directional dividers 78a, 78b can be beneficial for forming a three-layered substrate 10, such as described above and illustrated in FIG. 1A. Of course, it two layered substrates 110, 210 as described herein could be formed through a headbox 80 including a single z-directional divider 78a that provides a first z-directional layer 85a and a second z-directional layer 85b of the headbox 80. Further, in some embodiments, the headbox 80 need not include any z-directional dividers 78a, 78b, which may be particularly beneficial if further mixing of fibers and/or components within the headbox 80 is desired.

The headbox 80 can provide a resultant slurry 76 to a forming surface 94. The forming surface 94 can be a foraminous sheet, such as a woven belt or screen, or any other suitable surface for accepting the resultant slurry 76.

The apparatus 11 can also include a dewatering system 96 that can be configured to remove liquid from the resultant slurry 76 (e.g., forming fluid) on the forming surface 94. In some embodiments, the dewatering system 96 can be configured to provide a vacuum to the resultant slurry 76 to pull liquid from the resultant slurry 76, and in doing so, can turn the resultant slurry 76 including the plurality of fibers 20 and the component 44, if present, into an absorbent material 10. In some embodiments, the dewatering system 96 can begin dewatering on fibers and/or components as they are still within the headbox 80.

Dewatering systems 96 drawing liquid from the resultant slurry 76 can also unintentionally draw components 44 (such as particulate SAM) through the forming surface 94, and/or cause components 44 to become lodged in the forming surface 94. Not only can this cause substrates 10 to be formed that do not include intended amounts of the component 44, but components 44 becoming lodged in the forming surface 94 and/or being drawn through the forming surface 94 can cause processing issues, including, but not limited to, reduced dewatering and/or increased demands for drying of the resultant slurry 76, machine down-time for cleaning, and increased complexity for dewatered liquid by including such components 44. Forming an absorbent substrate 10 including components 44 in a fluid, such as foam forming, can exacerbate the problem of component 44 movement in the resultant slurry 76 in comparison to dry forming techniques, such as air-laid formation techniques or adhesive-based techniques.

Importantly, forming a containment layer 17 as part of the substrate 10, 210 that is directly against the forming surface 94 can help protect the components 44 of the substrate 12 (such as SAM in the absorbent layer 13). The containment layer 17 can protect the components 44 of the substrate 10 from the forming surface to help ensure the components 44 remain in the substrate 10, 210, or at least reduce the possibility for the components 44 to become lodged in the forming surface 94 or be drawn through the forming surface 94. Additionally, the containment layer 17 can help retain components 44 within the absorbent material 10, 210 as it is potentially transported for further processing and/or use in other products in which the absorbent material 10, 210 may be incorporated within, such as personal care absorbent articles. Forming the containment layer 17 inline as a layered composite with the absorbent layer 13 where at least some fibers of the containment layer 17 are mixed with at least some of the fibers of the absorbent layer 13 at the interface 19, eliminates the need for additional processing to form a composite absorbent substrate 10, 210, such as the use of adhesive to couple a separate containment layer 17 to an absorbent layer 13. Eliminating adhesive can result in reduced processing equipment and raw material cost, and can also lead to improved fluid handling properties of the absorbent substrate 10, 210. Additionally, forming a containment layer 17 as part of the substrate 10, 210 can also provide improved integrity and tensile strength for the absorbent material 10, 210, providing enhanced processing capability of the substrate 10, 210.

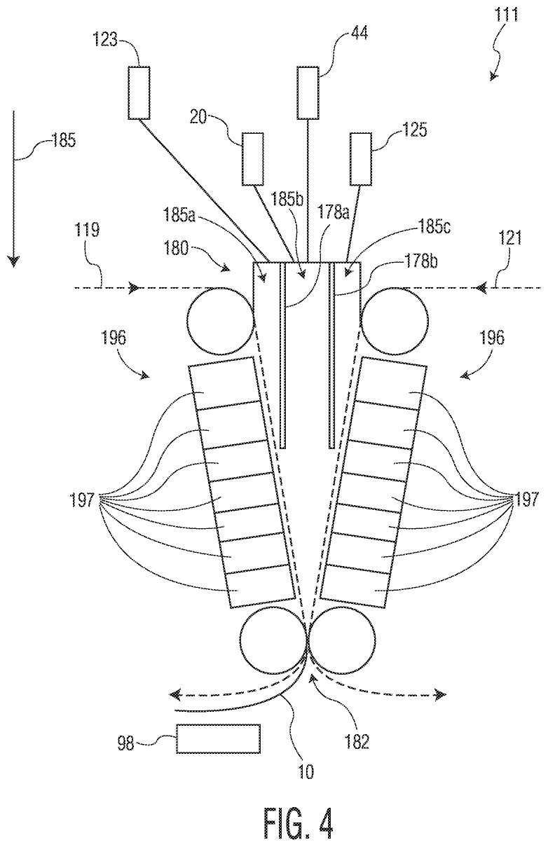

While the apparatus 11 and method described in FIG. 2 is one exemplary embodiment for forming an absorbent material 10, an alternative embodiment of an apparatus 111 and method of forming an absorbent material 10 is depicted in FIG. 4. The apparatus 111 of FIG. 4 can be used as part of a similar foam forming process as described above with respect to FIG. 2, however, the headbox 180 is a vertical twin former as is known in the art. The headbox 180 can include first and second foraminous elements 119, 121. The first and second foraminous elements 119, 121 can help define an interior volume of the headbox 180. The headbox 180 can include a first divider 178a and a second divider 178b that can provide first, second, and third z-directional layers 185a, 185b, 185c within the headbox 180 similar to the discussion above with the headbox 80 in FIG. 3, but the layers 185a, 185b, 185c in FIG. 4 are in a vertical orientation with respect to one another due to the vertical orientation of the headbox 180. The apparatus 111 can include a dewatering system 196 that can include a series of vacuum elements 197 disposed adjacent each foraminous element 119, 121.

In some embodiments, a first supply of fibers 20 can be supplied to the headbox 180, and in some embodiments, the first supply of fibers 20 can be in a foam. The supply of the fibers 20 can include at least some absorbent fibers. The supply of the component 44 can also be supplied directly to the headbox 180, and in some embodiments, the supply of the component 44 may be in a foam. The supply of the fibers 20 and component 44 can be delivered to the second z-directional layer 185b of the headbox 180. It is to be noted that in some embodiments, the second z-directional layer 185b of the headbox 180 may only be provided with the supply of the component 44 and not a supply of fibers 20. In some embodiments, a second supply of fibers 123 can be provided to the headbox 180, and in some embodiments, can be in a foam. The second supply of fibers 123 can be provided to the first z-directional layer 185a of the headbox 180. In some embodiments, a third supply of fibers 125 can be provided to the headbox 180, and in some embodiments, can be in a foam. The third supply of fibers 125 can be provided to the third z-directional layer 185c of the headbox 180. The fibers 20, 123, 125 and component 44 can be processed through the headbox 180 in a machine direction 185 towards the outlet 182 of the headbox 180 to provide an absorbent material 10, similar to the apparatus 11 described in FIG. 2.

The apparatuses 11, 111 as described herein can also include a drying system 98 to further dry and/or cure the absorbent material 10, 110, 210. The drying system 98 can apply heat to the absorbent material 10, such as by providing heated air in a through-air drying system.

In some embodiments, the apparatus 11, 111 can include a winding system 99 (as shown in FIG. 2) that can be configured to wind the absorbent material 10, 110, 210 in a roll fashion. In other embodiments, the apparatus 11, 111 can festoon the absorbent material 10, 110, 210, or collect the absorbent material 10, 110, 210 in any other suitable configuration, such as spooling.

### Foaming Fluid

The foam forming processes as described herein can include a foaming fluid. In some embodiments, the foaming fluid can comprise between about 85% to about 99.99% of the foam (by weight). In some embodiments, the foaming fluid used to make the foam can comprise at least about 85% of the foam (by weight). In certain embodiments, the foaming fluid can comprise between about 90% and about 99.9% % of the foam (by weight). In certain other embodiments, the foaming fluid can comprise between about 93% and 99.5% of the foam or even between about 95% and about 99.0% of the foam (by weight). In preferred embodiments, the foaming fluid can be water, however, it is contemplated that other processes may utilize other foaming fluids.

### Foaming Surfactant

The foam forming processes as described herein can utilize one or more surfactants. The fibers and surfactant, together with the foaming liquid and any additional components, can form a stable dispersion capable of substantially retaining a high degree of porosity for longer than the drying process. In this regard, the surfactant is selected so as to provide a foam having a foam half life of at least 2 minutes, more desirably at least 5 minutes, and most desirably at least 10 minutes. A foam half life can be a function of surfactant types, surfactant concentrations, foam compositions/solid level and mixing power/air content in a foam. The foaming surfactant used in the foam can be selected from one or more known in the art that are capable of providing the desired degree of foam stability. In this regard, the foaming surfactant can be selected from anionic, cationic, nonionic and amphoteric surfactants provided they, alone or in combination with other components, provide the necessary foam stability, or foam half life. As will be appreciated, more than one surfactant can be used, including different types of surfactants, as long as they are compatible, and more than one surfactant of the same type. For example, a combination of a cationic surfactant and a nonionic surfactant or a combination of an anionic surfactant and a nonionic surfactant may be used in some embodiments due to their compatibilities. However, in some embodiments, a combination of a cationic surfactant and an anionic surfactant may not be satisfactory to combine due to incompatibilities between the surfactants.

Anionic surfactants believed suitable for use with the present disclosure include, without limitation, anionic sulfate surfactants, alkyl ether sulfonates, alkylaryl sulfonates, or mixtures or combinations thereof. Examples of alkylaryl sulfonates include, without limitation, alkyl benzene sulfonic acids and their salts, dialkylbenzene disulfonic acids and their salts, dialkylbenzene sulfonic acids and their salts, alkylphenol sulfonic acids/condensed alkylphenol sulfonic acids and their salts, or mixture or combinations thereof. Examples of additional anionic surfactants believed suitable for use in the present disclosure include alkali metal sulforicinates, sulfonated glyceryl esters of fatty acids such as sulfonated monoglycerides of coconut oil acids, salts of sulfonated monovalent alcohol esters such as sodium oleylisethianate, metal soaps of fatty acids, amides of amino sulfonic acids such as the sodium salt of oleyl methyl tauride, sulfonated products of fatty acids nitriles such as palmitonitrile sulfonate, alkali metal alkyl sulfates such as sodium lauryl sulfate, ammonium lauryl sulfate or triethanolamine lauryl sulfate, ether sulfates having alkyl groups of 8 or more carbon atoms such as sodium lauryl ether sulfate, ammonium lauryl ether sulfate, sodium alkyl aryl ether sulfates, and ammonium alkyl aryl ether sulfates, sulphuric esters of polyoxyethylene alkyl ether, sodium salts, potassium salts, and amine salts of alkylnapthylsulfonic acid. Certain phosphate surfactants including phosphate esters such as sodium lauryl phosphate esters or those available from the Dow Chemical Company under the tradename TRITON are also believed suitable for use herewith. A particularly desired anionic surfactant is sodium dodecyl sulfate (SDS).

Cationic surfactants are also believed suitable for use with the present disclosure for manufacturing some embodiments of substrates. In some embodiments, such as those including superabsorbent material, cationic surfactants may be less preferable to use due to potential interaction between the cationic surfactant(s) and the superabsorbent material, which may be anionic. Foaming cationic surfactants include, without limitation, monocarbyl ammonium salts, dicarbyl ammonium salts, tricarbyl ammonium salts, monocarbyl phosphonium salts, dicarbyl phosphonium salts, tricarbyl phosphonium salts, carbylcarboxy salts, quaternary ammonium salts, imidazolines, ethoxylated amines, quaternary phospholipids and so forth. Examples of additional cationic surfactants include various fatty acid amines and amides and their derivatives, and the salts of the fatty acid amines and amides. Examples of aliphatic fatty acid amines include dodecylamine acetate, octadecylamine acetate, and acetates of the amines of tallow fatty acids, homologues of aromatic amines having fatty acids such as dodecylanalin, fatty amides derived from aliphatic diamines such as undecylimidazoline, fatty amides derived from aliphatic diamines such as undecylimidazoline, fatty amides derived from disubstituted amines such as oleylaminodiethylamine, derivatives of ethylene diamine, quaternary ammonium compounds and their salts which are exemplified by tallow trimethyl ammonium chloride, dioctadecyldimethyl ammonium chloride, didodecyldimethyl ammonium chloride, dihexadecyl ammonium chloride, alkyltrimethylammonium hydroxides, dioctadecyldimethylammonium hydroxide, tallow trimethylammonium hydroxide, trimethylammonium hydroxide, methylpolyoxyethylene cocoammonium chloride, and dipalmityl hydroxyethylammonium methosulfate, amide derivatives of amino alcohols such as beta-hydroxylethylstearylamide, and amine salts of long chain fatty acids. Further examples of cationic surfactants believed suitable for use with the present disclosure include benzalkonium chloride, benzethonium chloride, cetrimonium bromide, distearyldimethylammonium chloride, tetramethylammonium hydroxide, and so forth.

Nonionic surfactants believed suitable for use in the present disclosure include, without limitation, condensates of ethylene oxide with a long chain fatty alcohol or fatty acid, condensates of ethylene oxide with an amine or an amide, condensation products of ethylene and propylene oxides, fatty acid alkylol amide and fatty amine oxides. Various additional examples of non-ionic surfactants include stearyl alcohol, sorbitan monostearate, octyl glucoside, octaethylene glycol monododecyl ether, lauryl glucoside, cetyl alcohol, cocamide MEA, monolaurin, polyoxyalkylene alkyl ethers such as polyethylene glycol long chain (12-14C) alkyl ether, polyoxyalkylene sorbitan ethers, polyoxyalkylene alkoxylate esters, polyoxyalkylene alkylphenol ethers, ethylene glycol propylene glycol copolymers, polyvinyl alcohol, alkylpolysaccharides, polyethylene glycol sorbitan monooleate, octylphenol ethylene oxide, and so forth. Non-ionic surfactants may be preferable when foam forming absorbent materials 10, 110, 210 with SAM. If there is residual ionic surfactant, the increase in ionic strength in the insult can reduce SAM swelling for use of the absorbent materials 10, 110, 210 in personal care absorbent articles.

The foaming surfactant can be used in varying amounts as necessary to achieve the desired foam stability and air-content in the foam. In certain embodiments, the foaming surfactant can comprise between about 0.005% and about 5% of the foam (by weight). In certain embodiments the foaming surfactant can comprise between about 0.05% and about 3% of the foam or even between about 0.05% and about 2% of the foam (by weight).

Fibers

As noted above, the apparatus 11, 111 and methods described herein can include providing a fibers from a supply of fibers 20, 37, 70, 123, 125. In some embodiments, the fibers can be suspending in a fluid supply 16, 28, 33, 68 that can be a foam. The foam suspension of fibers can provide one or more supply of fibers. As described above, fibers utilized herein can include natural fibers and/or synthetic fibers. In some embodiments, a fiber supply 20, 37, 70, 123, 125 can include only natural fibers or only synthetic fibers. In other embodiments, a fiber supply 20, 37, 70, 123, 125 can include a mixture of natural fibers and synthetic fibers. Some fibers being utilized herein can be absorbent, whereas other fibers utilized herein can be non-absorbent. Non-absorbent fibers can provide features for the substrates that are formed from the methods and apparatuses described herein, such as improved intake or distribution of fluids.

In some embodiments, the total content of fibers, can comprise between about 0.01% to about 10% of the foam (by weight), and in some embodiments between about 0.1% to about 5% of the foam (by weight).

Binder

In some embodiments, a fluid supply 16, 28, 33, 68 can include binder materials (as described above) that can be provided along with or independent of the supply of the fibers 20, 37, 70, 123, 125 or the supply of the component 44.

Binders can additionally or alternatively be provided in a liquid form, such as latex emulsions, and can comprise between about 0% and about 10% of the foam (by weight). In certain embodiments the non-fibrous binder can comprise between about 0.1% and 10% of the foam (by weight) or even between about 0.2% and about 5% or even between about 0.5% and about 2% of the foam (by weight).

Binder fibers, when used, may be added proportionally to the other components to achieve the desired fiber ratios and structure while maintaining the total solids content of the foam below the amounts stated above. As an example, in some embodiments, binder fibers can comprise between about 0% and about 50% of the total fiber weight, and more preferably, between about 5% to about 40% of the total fiber weight in some embodiments.

Foam Stabilizers

In some embodiments, if a fluid supply 16, 28, 33, 68 is configured as a foam the foam may optionally also include one or more foam stabilizers known in the art and that are compatible with the components of the foam and further do not interfere with the hydrogen bonding as between the cellulosic fibers. Foam stabilizing agents believed suitable for use in the present disclosure, without limitation, one or more zwitterionic compounds, amine oxides, alkylated polyalkylene oxides, or mixture or combinations thereof. Specific examples of foam stabilizers includes, without limitation, cocoamine oxide, isononyldimethylamine oxide, n-dodecyldimethylamine oxide, and so forth.

In some embodiments, if utilized, the foam stabilizer can comprise between about 0.01% and about 2% of the foam (by weight). In certain embodiments, the foam stabilizer can comprise between about 0.05% and 1% of the foam or even between about 0.1 and about 0.5% of the foam (by weight).

Components

As mentioned above, foam forming processes can include adding one or more components 44 as additional additives that will be incorporated into the absorbent material 10, 110, 210, such as SAM. In some embodiments incorporating SAM, the SAM can comprise between about 0% and about 40% of the foam (by weight). In certain embodiments, SAM can comprise between about 1% and about 30% of the foam (by weight) or even between about 10% and about 30% of the foam (by weight).

If used, wet and dry strength additives can comprise between about 0.01 and about 5% of the dry weight of cellulose fibers. In certain embodiments, the strength additives can comprise between about 0.05% and about 2% of the dry weight of cellulose fibers or even between about 0.1% and about 1% of the dry weight of cellulose fibers.

When employed, miscellaneous components that may also be used in the absorbent material (as described above, such as, pigments, anti-microbial agents, etc.) can desirably comprise less than about 2% of the foam (by weight) and still more desirably less than about 1% of the foam (by weight) and even less than about 0.5% of the foam (by weight).

In some embodiments, the solids content, including the fibers or particulates contained herein, desirably comprise no more than about 40% of the foam. In certain embodiments the cellulosic fibers can comprise between about 0.1% and about 5% of the foam or between about 0.2 and about 4% of the foam or even between about 0.5% and about 2% of the foam.

The methods and apparatuses 11, 111 as described herein can be beneficial for forming one or more absorbent materials 10, 110, 210. The absorbent materials 10, 110, 210 as described herein can be useful as components of personal care products. For example, in one embodiment, the absorbent material 10, 110, 210 as described herein can be an absorbent composite for personal care absorbent articles. The multi-layer absorbent materials 10, 110, 210 as described herein may also be beneficial for using in other products, such as, but not limited to facial tissues, bath tissues, wipes, and wipers.

### Examples

Extensive experimental testing was conducted to create over 100 different absorbent materials through a foam forming process as described above. Table 1 provides a listing of the exemplary codes created for absorbent materials 110 including an intake layer 12 and absorbent layer 13. The surfactant used in foam forming to produce the experimental codes was Stantex H215 UP, a non-ionic surfactant commercially produced by Pulcra Chemicals. The PET Curly Fibers used were 6 denier fiber diameter and 6 mm in fiber length with 10 crimps per cm, manufactured by William Barnet Inc. The T255 Binder Fibers used were a PE/PET sheath/core structure and had a 2.2 dtex fiber diameter and a 6 mm fiber length, manufactured by Trevira. The CMC 535 Pulp Fiber used in the experimental codes was a crosslinked pulp fiber, manufactured by International Paper. NBSK is a Northern Bleached Softwood Kraft, a commercial northern softwood pulp fiber. SBSK is a Southern Bleached Softwood Kraft, a commercial southern softwood pulp fiber. SAM used in the experimental codes was commercially available SAM SXM 5660, manufactured by Evonik. In Table 1, an asterisk is being used to denote properties that were not measured/calculated.

TABLE 1
Exemplary Absorbent Materials

| | Targeted Basis Weight (gsm) | | | | | | | | | | Measured/Calculated | | | |
|---|---|---|---|---|---|---|---|---|---|---|---|---|---|---|
| | Intake Layer | | | Absorbent Layer | | | | | | | | % | % | % |
| | PET | CMC | T255 | T255 | | | | CMC | PET | | Approx | SAM in | binder in | binder in |
| Code | Curly | 535 | Binder | Binder | | | | 535 | Curly | | SAM | absorbent | absorbent | intake |
| No. | Fiber | fiber | Fiber | Fiber | NBSK | eucalyptus | SBSK | Fiber | Fiber | SAM | (gsm) | layer | layer | layer |
| 1 | 0 | 0 | 0 | 60 | 40 | 0 | 0 | 0 | 0 | 500 | 414 | 81% | 12% | 0% |
| 2 | 0 | 0 | 0 | 60 | 40 | 0 | 0 | 0 | 0 | 500 | 350 | 78% | 13% | 0% |
| 3 | 64 | 0 | 28 | 55 | 27 | 0 | 0 | 0 | 0 | 400 | 298 | 78% | 14% | 30% |
| 4 | 69 | 0 | 31 | 100 | 0 | 0 | 0 | 210 | 0 | 490 | 300 | 49% | 16% | 31% |
| 5 | 69 | 0 | 31 | 100 | 40 | 0 | 0 | 170 | 0 | 490 | 399 | 56% | 14% | 31% |
| 6 | 0 | 0 | 0 | 0 | 140 | 0 | 0 | 0 | 0 | 25 | 81 | 37% | 0% | |
| 7 | 0 | 80 | 20 | 0 | 140 | 0 | 0 | 0 | 0 | 25 | 103 | 42% | 0% | 20% |
| 8 | 73 | 0 | 19 | 62 | 0 | 0 | 0 | 30 | 0 | 500 | 0 | * | * | 21% |
| 9 | 73 | 0 | 19 | 62 | 0 | 0 | 0 | 30 | 0 | 500 | 336 | 79% | 14% | 21% |
| 10 | 73 | 0 | 19 | 62 | 0 | 0 | 0 | 30 | 0 | 500 | 370 | 80% | 13% | 21% |
| 11 | 73 | 0 | 19 | 62 | 15 | 0 | 0 | 15 | 0 | 500 | 340 | 79% | 14% | 21% |
| 12 | 31 | 0 | 9 | 62 | 15 | 0 | 0 | 15 | 0 | 500 | 280 | 75% | 17% | 23% |
| 13 | 0 | 0 | 0 | 62 | 15 | 0 | 0 | 15 | 0 | 500 | 300 | 77% | 16% | 0% |
| 14 | 31 | 0 | 9 | 62 | 15 | 0 | 0 | 15 | 0 | 500 | 388 | 81% | 13% | 23% |
| 15 | 31 | 0 | 9 | 45.4 | 23.3 | 0 | 0 | 23.3 | 0 | 500 | 450 | 83% | 8% | 23% |
| 16 | 46 | 0 | 14 | 62 | 15 | 0 | 0 | 15 | 0 | 500 | * | * | * | 23% |
| 17 | 31 | 0 | 9 | 45.4 | 46.6 | 0 | 0 | 0 | 0 | 500 | 454 | 83% | 8% | 23% |
| 18 | 73 | 0 | 19 | 62 | 0 | 0 | 0 | 0 | 0 | 500 | 421 | 87 | 13% | 21% |
| 19 | 26 | 0 | 14 | 20 | 0 | 0 | 0 | 50 | 30 | 0 | 0 | 0% | 20% | 35% |
| 20 | 26 | 0 | 14 | 20 | 0 | 0 | 0 | 50 | 30 | 500 | 450 | 87% | 4% | 35% |
| 21 | 0 | 0 | 0 | 62 | 30 | 0 | 0 | 0 | 0 | 500 | 400 | 81% | 13% | 0% |
| 22 | 26 | 0 | 14 | 46.0 | 46.0 | 0 | 0 | 0.0 | 0.0 | 475 | 558.7 | 86% | 7% | 35% |
| 23 | 26 | 0 | 14 | 46.0 | 46.0 | 0 | 0 | 0.0 | 0.0 | 475 | 160.5 | 64% | 18% | 35% |
| 24 | 26 | 0 | 14 | 46.0 | 46.0 | 0 | 0 | 0.0 | 0.0 | 475 | 429.5 | 82% | 9% | 35% |
| 25 | 26 | 0 | 14 | 46.0 | 46.0 | 0 | 0 | 0.0 | 0.0 | 475 | 510.6 | 85% | 8% | 35% |
| 26 | 31 | 0 | 9 | 46.0 | 46.0 | 0 | 0 | 0.0 | 0.0 | 475 | 473.2 | 84% | 8% | 23% |
| 27 | 0 | 0 | 0 | 46.0 | 46.0 | 0 | 0 | 0.0 | 0.0 | 475 | 432.7 | 82% | 9% | 0% |
| 28 | 14 | 0 | 26 | 30.0 | 0.0 | 0 | 0 | 40.0 | 30.0 | 475 | 446.4 | 86% | 5% | 65% |
| 29 | 14 | 0 | 26 | 62.0 | 15.0 | 0 | 0 | 15.0 | 0.0 | 475 | 560.6 | 86% | 10% | 65% |
| 30 | 26 | 0 | 14 | 46.0 | 23.0 | 0 | 0 | 23.0 | 0.0 | 475 | 406.3 | 82% | 9% | 35% |
| 31 | 31 | 0 | 9 | 46.0 | 46.0 | 0 | 0 | 0.0 | 0.0 | 475 | 482.4 | 84% | 8% | 23% |
| 32 | 0 | 0 | 0 | 46.0 | 46.0 | 0 | 0 | 0.0 | 0.0 | 475 | 480.3 | 84% | 8% | 0% |
| 33 | 31 | 0 | 9 | 62.0 | 15.0 | 0 | 0 | 15.0 | 0.0 | 475 | 491.3 | 84% | 11% | 23% |
| 34 | 0 | 0 | 0 | 62.0 | 15.0 | 0 | 0 | 15.0 | 0.0 | 475 | 531.5 | 85% | 10% | 0% |
| 35 | 0 | 0 | 0 | 46.0 | 46.0 | 0 | 0 | 0.0 | 0.0 | 475 | 410.1 | 82% | 9% | 0% |
| 36 | 31 | 0 | 9 | 62.0 | 15.0 | 0 | 0 | 16.0 | 0.0 | 475 | 418.3 | 82% | 12% | 23% |
| 37 | 26 | 0 | 14 | 30.0 | 0.0 | 0 | 0 | 40.0 | 30.0 | 400 | * | * | * | 35% |
| 38 | 26 | 0 | 14 | 30.0 | 0.0 | 0 | 0 | 40.0 | 30.0 | 400 | * | * | * | 35% |
| 39 | 26 | 0 | 14 | 30.0 | 0.0 | 0 | 0 | 40.0 | 30.0 | 400 | 414.9 | 86% | 6% | 35% |
| 40 | 26 | 0 | 14 | 30.0 | 0.0 | 0 | 0 | 40.0 | 30.0 | 400 | 514 | 88% | 5% | 35% |
| 41 | 26 | 0 | 14 | 30.0 | 0.0 | 0 | 0 | 40.0 | 30.0 | 400 | 408.0 | 85% | 6% | 35% |
| 42 | 26 | 0 | 14 | 30.0 | 0.0 | 0 | 0 | 40.0 | 30.0 | 400 | 496.8 | 88% | 5% | 35% |
| 43 | 26 | 0 | 14 | 30.0 | 0.0 | 0 | 0 | 40.0 | 30.0 | 400 | 462.6 | 87% | 5% | 35% |
| 44 | 6.5 | 0 | 3.5 | 7.5 | 0.0 | 0 | 0 | 10.0 | 7.5 | 100 | 127.2 | 88% | 5% | 35% |
| 45 | 26 | 0 | 14 | 30.0 | 0.0 | 0 | 0 | 40.0 | 30.0 | 400 | 478.4 | 87% | 5% | 35% |
| 46 | 26 | 0 | 14 | 30.0 | 0.0 | 0 | 0 | 40.0 | 30.0 | 400 | 355.3 | 84% | 7% | 35% |
| 47 | 6.5 | 0 | 3.5 | 7.5 | 0.0 | 0 | 0 | 10.0 | 7.5 | 100 | 125.8 | 88% | 5% | 35% |
| 48 | 6.5 | 0 | 3.5 | 7.5 | 0.0 | 0 | 0 | 10.0 | 7.5 | 100 | 119.4 | 87% | 5% | 35% |
| 49 | 6.5 | 0 | 3.5 | 7.5 | 0.0 | 0 | 0 | 10.0 | 7.5 | 100 | 118.8 | 87% | 5% | 35% |
| 50 | 6.5 | 0 | 3.5 | 7.5 | 0.0 | 0 | 0 | 10.0 | 7.5 | 100 | 131.6 | 88% | 5% | 35% |
| 51 | 13 | 0 | 7 | 15.0 | 0.0 | 0 | 0 | 20.0 | 15.0 | 200 | 411.0 | 92% | 3% | 35% |
| 52 | 13 | 0 | 7 | 15.0 | 0.0 | 0 | 0 | 20.0 | 15.0 | 200 | 121.0 | 78% | 9% | 35% |
| 53 | 13 | 0 | 7 | 15.0 | 0.0 | 0 | 0 | 20.0 | 15.0 | 200 | 275.0 | 89% | 5% | 35% |
| 54 | 6.5 | 0 | 3.5 | 7.5 | 0.0 | 0 | 0 | 10.0 | 7.5 | 100 | 140.0 | 89% | 5% | 35% |
| 55 | 26 | 0 | 14 | 30.0 | 0.0 | 0 | 0 | 40.0 | 30.0 | 400 | 535.0 | 88% | 5% | 35% |
| 56 | 26 | 0 | 14 | 30.0 | 0.0 | 0 | 0 | 40.0 | 30.0 | 400 | 536.0 | 88% | 5% | 35% |
| 57 | 26 | 0 | 14 | 30.0 | 0.0 | 0 | 0 | 40.0 | 30.0 | 400 | 543.0 | 89% | 5% | 35% |
| 58 | 0 | 0 | 0 | 22.5 | 0.0 | 0 | 0 | 30 | 22.5 | 0 | 0 | 0% | 30% | 0% |
| 59 | 0 | 0 | 0 | 22.5 | 0.0 | 0 | 0 | 30 | 22.5 | 100 | 97.8 | 65% | 13% | 0% |
| 60 | 0 | 0 | 0 | 22.5 | 0.0 | 0 | 0 | 30 | 22.5 | 0 | 0 | 0% | 30% | 0% |
| 61 | 0 | 0 | 0 | 22.5 | 0.0 | 0 | 0 | 30 | 22.5 | 100 | 97.80 | 65% | 13% | 0% |
| 62 | 0 | 0 | 0 | 37.5 | 0.0 | 0 | 0 | 50 | 37.5 | 0 | 0 | 0% | 30% | 0% |
| 63 | 0 | 0 | 0 | 37.5 | 0.0 | 0 | 0 | 50 | 37.5 | 100 | 93.0 | 52% | 17% | 0% |
| 64 | 9 | 12 | 9 | 22.5 | 0.0 | 0 | 0 | 30 | 22.5 | 0 | 0 | 0% | 30% | 30% |
| 65 | 9 | 12 | 9 | 22.5 | 0.0 | 0 | 0 | 30 | 22.5 | 100 | 96.4 | 65% | 13% | 30% |
| 66 | 9 | 12 | 9 | 22.5 | 0.0 | 0 | 0 | 30 | 22.5 | 200 | 191.6 | 78% | 8% | 30% |
| 67 | 9 | 12 | 9 | 22.5 | 0.0 | 0 | 0 | 30 | 22.5 | 300 | 263.8 | 83% | 7% | 30% |
| 68 | 3 | 4 | 3 | 22.5 | 0.0 | 0 | 0 | 30 | 22.5 | 0 | 0 | 0% | 30% | 30% |
| 69 | 3 | 4 | 3 | 22.5 | 0.0 | 0 | 0 | 30 | 22.5 | 200 | * | * | * | 30% |
| 70 | 3 | 4 | 3 | 22.5 | 0.0 | 0 | 0 | 30 | 22.5 | 0 | 0 | 0% | 30% | 30% |
| 71 | 3 | 4 | 3 | 22.5 | 0.0 | 0 | 0 | 30 | 22.5 | 300 | 248.6 | 83% | 7% | 30% |
| 72 | 3 | 4 | 3 | 22.5 | 0.0 | 0 | 0 | 30 | 22.5 | 400 | 325.5 | 86% | 6% | 30% |
| 73 | 3 | 4 | 3 | 22.5 | 0.0 | 0 | 0 | 30 | 22.5 | 0 | 0 | 0% | 30% | 30% |
| 74 | 3 | 4 | 3 | 22.5 | 0.0 | 0 | 0 | 30 | 22.5 | 500 | 394.3 | 88% | 5% | 30% |
| 75 | 3 | 4 | 3 | 22.5 | 0.0 | 0 | 0 | 30 | 22.5 | 0 | 0 | 0% | 30% | 30% |
| 76 | 3 | 4 | 3 | 22.5 | 0.0 | 0 | 0 | 30 | 22.5 | 500 | 394.9 | 88% | 5% | 30% |
| 77 | 3 | 4 | 3 | 22.5 | 0.0 | 0 | 0 | 30 | 22.5 | 550 | 425.9 | 89% | 4% | 30% |
| 78 | 3 | 4 | 3 | 22.5 | 0.0 | 0 | 0 | 30 | 22.5 | 600 | 404 | 88% | 5% | 30% |
| 79 | 3 | 4 | 3 | 22.5 | 0.0 | 0 | 0 | 30 | 22.5 | 650 | * | * | * | 30% |
| 80 | 26 | 0 | 14 | 22.5 | 0.0 | 0 | 0 | 30 | 22.5 | 0 | 0 | 0% | 30% | 35% |
| 81 | 26 | 0 | 14 | 22.5 | 0.0 | 0 | 0 | 30 | 22.5 | 200 | 228.6 | 81% | 7% | 35% |
| 82 | 26 | 0 | 14 | 22.5 | 0.0 | 0 | 0 | 30 | 22.5 | 300 | 339.6 | 87% | 5% | 35% |
| 83 | 26 | 0 | 14 | 22.5 | 0.0 | 0 | 0 | 30 | 22.5 | 400 | 450.6 | 90% | 4% | 35% |
| 84 | 26 | 0 | 14 | 22.5 | 0.0 | 0 | 0 | 30 | 22.5 | 500 | 578.3 | 92% | 3% | 35% |
| 85 | 26 | 0 | 14 | 22.5 | 0.0 | 0 | 0 | 30 | 22.5 | 550 | 561 | 91% | 4% | 35% |
| 86 | 26 | 0 | 14 | 22.5 | 0.0 | 0 | 0 | 30 | 22.5 | 550 | 575.8 | 92% | 3% | 35% |
| 87 | 26 | 0 | 14 | 37.5 | 0.0 | 0 | 18.75 | 18.8 | 0 | 0 | 0 | 0% | 50% | 35% |
| 88 | 26 | 0 | 14 | 37.5 | 0.0 | 0 | 18.75 | 18.8 | 0 | 100 | 125.2 | 63% | 19% | 35% |
| 89 | 26 | 0 | 14 | 37.5 | 0.0 | 0 | 18.75 | 18.8 | 0 | 200 | 218.9 | 74% | 13% | 35% |
| 90 | 26 | 0 | 14 | 37.5 | 0.0 | 0 | 18.75 | 18.8 | 0 | 300 | 302.2 | 80% | 10% | 35% |
| 91 | 26 | 0 | 14 | 37.5 | 0.0 | 0 | 18.75 | 18.8 | 0 | 400 | * | * | * | 35% |
| 92 | 26 | 0 | 14 | 20 | 0.0 | 0 | 10 | 10 | 0 | 0 | 0 | 0% | 50% | 35% |
| 93 | 26 | 0 | 14 | 20 | 0.0 | 0 | 10 | 10 | 0 | 150 | 210 | 84% | 8% | 35% |
| 94 | 26 | 0 | 14 | 20 | 0.0 | 0 | 10 | 10 | 0 | 200 | * | * | * | 35% |
| 95 | 26 | 0 | 14 | 20 | 0.0 | 0 | 10 | 10 | 0 | 150 | * | * | * | 35% |
| 96 | 3 | 4 | 3 | 12 | 0.0 | 0 | 0 | 16 | 12 | 150 | 168.0 | 86% | 6% | 30% |
| 97 | 3 | 4 | 3 | 12 | 0.0 | 0 | 0 | 16 | 12 | 150 | 189.4 | 87% | 5% | 30% |
| 98 | 3 | 4 | 3 | 12 | 0.0 | 0 | 0 | 16 | 12 | 150 | 153.1 | 85% | 6% | 30% |
| 99 | 3 | 4 | 3 | 12 | 0.0 | 0 | 0 | 16 | 12 | 250 | 284.7 | 91% | 4% | 30% |
| 100 | 26 | 0 | 14 | 24 | 0.0 | 0 | 0 | 32 | 24 | 150 | 192.5 | 77% | 9% | 35% |
| 101 | 32.5 | 0 | 17.5 | 22.5 | 0 | 0 | 0 | 30 | 22.5 | 400 | 408.3 | 86% | 4% | 35% |
| 102 | 32.5 | 0 | 17.5 | 22.5 | 0 | 0 | 0 | 30 | 22.5 | 400 | 371.4 | 81% | 6% | 35% |
| 103 | 32.5 | 0 | 17.5 | 22.5 | 0 | 0 | 0 | 30 | 22.5 | 400 | 333.9 | 80% | 6% | 35% |
| 104 | 39 | 0 | 21 | 22.5 | 18.75 | 0 | 0 | 18.75 | 15 | 400 | 427.5 | 88% | 4% | 20% |
| 105 | 52 | 0 | 28 | 22.5 | 18.75 | 0 | 0 | 18.75 | 15 | 400 | * | * | * | 35% |
| 106 | 48 | 0 | 12 | 30 | 0 | 0 | 0 | 20 | 50 | 345 | 358.7 | 80% | 6% | 35% |
| 107 | 26 | 0 | 14 | 55 | 22 | 0 | 0 | 11 | 22 | 345 | * | * | * | 35% |
| 108 | 26 | 0 | 14 | 8 | 5 | 0 | 0 | 5 | 2 | 345 | 306.0 | 92% | 3% | 35% |
| 109 | 26 | 0 | 14 | 16 | 10 | 0 | 0 | 10 | 4 | 345 | 333.6 | 88% | 5% | 35% |
| 110 | 26 | 0 | 14 | 4 | 2.5 | 0 | 0 | 2.5 | 1 | 345 | 302.6 | 96% | 2% | 35% |
| 111 | 26 | 0 | 14 | 8 | 5 | 0 | 0 | 5 | 2 | 345 | * | * | * | 35% |
| 112 | 26 | 0 | 14 | 4 | 0 | 2.5 | 0 | 2.5 | 1 | 345 | 311.2 | 97% | 1% | 35% |
| 113 | 26 | 0 | 14 | 8 | 0 | 5 | 0 | 5 | 2 | 345 | 306.9 | 95% | 2% | 35% |
| 114 | 26 | 0 | 14 | 8 | 0 | 5 | 0 | 5 | 2 | 345 | 280.5 | 93% | 3% | 35% |
| 115 | 26 | 0 | 14 | 22.5 | 0 | 0 | 0 | 30 | 22.5 | 400 | 378 | 83% | 5% | 35% |
| 116 | 32 | 0 | 8 | 8 | 5 | 0 | 0 | 5 | 2 | 345 | 361 | 95% | 2% | 20% |
| 117 | 48 | 0 | 12 | 8 | 5 | 0 | 0 | 5 | 2 | 400 | 357 | 95% | 2% | 20% |
| 118 | 26 | 0 | 14 | 8 | 20 | 0 | 0 | 0 | 12 | 400 | 380 | 90% | 2% | 35% |

As can be seen in the codes above, many absorbent materials codes were successfully created through a foam-forming process that included greater than 80% SAM in the absorbent layer 13. The exemplary codes described in Table 1 above were then tested for various physical characteristics described in the Test Methods Section herein, including saturation capacity (Sat. Cap.) under the Saturation Capacity Test, first, second, and third intake times under the FIUP Test, and rewet under the Rewet Test. Experimental codes were also measured for dry thickness and wet thickness.

Three controls were tested for comparative purposes for the experimental codes. Control 1 was an exemplary absorbent composite construction of commercially available Poise® Ultra Thin Moderate 4-drop Regular Pads (manufactured by Kimberly-Clark Corporation in 2020) having a width of 62 mm, a length of 215 mm, and a basis weight of 561 gsm. Control 2 was an exemplary absorbent composite construction of commercially available Always Discreet® Moderate 4-drop Regular pads (manufactured by Proctor & Gamble in 2019) having a width of 59 mm and a length of 215 mm. Control 3 was an exemplary absorbent composite construction of commercially available Always Discreet® Moderate 4-drop Regular pads (manufactured by Proctor & Gamble in April of 2020) having a width of 59 mm and a length of 215 mm.

TABLE 2
Performance Testing for Exemplary Absorbent Materials

| Code | Sat. cap. | Dry thick | Wet thick | 1st | 2nd | 3rd | Rewet | |
|---|---|---|---|---|---|---|---|---|
| No. | (g) | (mm) | (mm) | intake (s) | intake (s) | intake (s) | (g) | Embossed |
| 1 | Not tested because intake layer was non-wettable | | | | | | | |
| 2 | Not tested because intake layer was non-wettable | | | | | | | |
| 3 | 113 | Not tested because intake layer was non-wettable | | | | | | |
| 4 | Not tested because intake layer was non-wettable | | | | | | | |
| 5 | Not tested because intake layer was non-wettable | | | | | | | |
| 6 | Not tested because intake layer was non-wettable | | | | | | | |
| 7 | Not tested because intake layer was non-wettable | | | | | | | |
| 8 | Not tested - aborted due to sheet break | | | | | | | |
| 9 | Not tested - SAM level too low with respect to target | | | | | | | |
| 10 | 125 | 9.42 | 14.4 | 7.5 | 13.3 | 25.6 | 4.65 | no |
| 10 | 168 | 9.25 | 15.6 | 7.4 | 14.5 | 25.2 | 0.6 | no |
| 10 | 152 | 6.88 | 14.5 | 8.8 | 18.1 | 31 | 0.33 | yes |
| 11 | Not tested - initial screening indicated very high rewet. | | | | | | | |
| 12 | Not tested - SAM level too low with respect to target | | | | | | | |
| 13 | Not tested - SAM level too low with respect to target | | | | | | | |
| 14 | 133 | 6.98 | 12.1 | 9.8 | 25.6 | 50.4 | 0.22 | no |
| 14 | 156 | 7.02 | 12.9 | 9.2 | 22.5 | 50.8 | 0.15 | no |
| 14 | 163 | 6.23 | 13.1 | 10.6 | 31 | 69.8 | 0.11 | yes |
| 15 | 146 | 7.54 | 14 | 7.1 | 27.2 | 43.7 | 0.242 | no |
| 15 | 200 | 7.94 | 15.7 | 8.6 | 34.4 | 78.7 | 0.07 | no |
| 15 | 170 | 6.3 | 14.6 | 14.1 | 33.7 | 71.5 | 0.13 | yes |
| 16 | Not tested | | | | | | | |
| 17 | 128 | 6.37 | 11.6 | 10.5 | 27.2 | 60 | 0.68 | no |
| 17 | 207 | 6.95 | 14.2 | 8.9 | 26.6 | 64.6 | 0.07 | no |
| 17 | 157 | 4.82 | 12.8 | 18.7 | 59.1 | 118 | 0.08 | yes |
| 18 | Not tested - initial screening indicated very high intake time | | | | | | | |
| 19 | Not tested - no SAM | | | | | | | |
| 20 | 173 | 7.75 | 14.4 | 8.3 | 26.8 | 61.8 | 0.1 | no |
| 20 | 208 | 7.79 | 15.8 | 8.1 | 22.9 | 56.4 | 0.07 | no |
| 20 | 165 | 5.94 | 15 | 12.5 | 41.7 | 97.7 | 0.08 | yes |
| 21 | 139 | 5.2 | 10.7 | 16 | 71 | 148 | 0.16 | no |
| 22 | Not tested - smaller batch made to test process conditions | | | | | | | |
| 23 | Not tested - smaller batch made to test process conditions | | | | | | | |
| 24 | Not tested - smaller batch made to test process conditions | | | | | | | |
| 25 | 121 | 5.65 | 10.9 | 13.9 | 29.5 | 54.8 | 0.1 | yes |
| 26 | 124 | 5.94 | 11.4 | 10.8 | 28.1 | 52 | 0.27 | yes |
| 26 | 176 | 5.6 | 12.5 | 13.5 | 33.8 | 66.1 | 0.12 | yes |
| 27 | Not tested | | | | | | | |
| 28 | 138 | 6.15 | 12.1 | 13.3 | 42.8 | 101 | 0.14 | yes |
| 29 | Not tested - initial screening indicated intake time is high | | | | | | | |
| 30 | Not tested | | | | | | | |
| 31 | 154 | 5.3 | 11.4 | 14.8 | 33.8 | 67.9 | 0.14 | yes |
| 31 | 185 | 5.6 | 11.7 | 13.1 | 32.9 | 70.6 | 0.12 | yes |
| 32 | 117 | 4.76 | 10.8 | 17.2 | 67.3 | 133 | 0.19 | yes |
| 33 | 115 | 6.03 | 12.1 | 11.8 | 60.1 | 77.4 | 0.13 | yes |
| 33 | 159 | 6.1 | 11.5 | 9 | 25.3 | 47.5 | 0.18 | yes |
| 34 | 127 | 5.91 | 11.6 | 13 | 73.5 | 135 | 0.13 | yes |
| 34 | 160 | 6.4 | 12.1 | 10.7 | 70.2 | 130.1 | 0.17 | yes |
| 35 | 118 | 5.46 | 11.8 | 14.1 | 50.3 | 97.5 | 0.12 | yes |
| 35 | 159 | 5.7 | 13.6 | 14 | 59.5 | 117.6 | 0.09 | yes |
| 36 | 135 | 6.32 | 12.2 | 11.6 | 45.8 | 79 | 0.11 | yes |
| 37 | Not tested | | | | | | | |
| 38 | Not tested | | | | | | | |
| 39 | Not tested | | | | | | | |
| 40 | Not tested | | | | | | | |
| 41 | Not tested | | | | | | | |
| 42 | Not tested | | | | | | | |
| 43 | Not tested | | | | | | | |
| 44 | Not tested | | | | | | | |
| 45 | Not tested | | | | | | | |
| 46 | Not tested | | | | | | | |
| 47 | Not tested | | | | | | | |
| 48 | Not tested | | | | | | | |
| 49 | Not tested | | | | | | | |
| 50 | Not tested | | | | | | | |
| 51 | Not tested | | | | | | | |
| 52 | Not tested | | | | | | | |
| 53 | Not tested | | | | | | | |
| 54 | Not tested | | | | | | | |
| 55 | Not tested | | | | | | | |
| 56 | Not tested | | | | | | | |
| 57 | Not tested | | | | | | | |
| 58 | Not tested - process check | | | | | | | |
| 59 | Not tested - process check | | | | | | | |
| 60 | Not tested - process check | | | | | | | |
| 61 | Not tested - process check | | | | | | | |
| 62 | Not tested - process check | | | | | | | |
| 63 | Not tested - process check | | | | | | | |
| 64 | Not tested - process check | | | | | | | |
| 65 | Not tested - process check | | | | | | | |
| 66 | Not tested - process check | | | | | | | |
| 67 | Not tested - process check | | | | | | | |
| 68 | Not tested - SAM too low with respect to target | | | | | | | |
| 69 | Not tested - SAM too low with respect to target | | | | | | | |
| 70 | Not tested - no SAM reference | | | | | | | |
| 71 | 111 | 5.97 | 10.76 | 15.15 | 50.86 | 163.17 | 0.154 | no |
| 72 | 131 | 6.81 | 12.89 | 11.81 | 43.23 | 117.38 | 0.131 | no |
| 73 | Not tested - no SAM reference | | | | | | | |
| 74 | Not tested | | | | | | | |
| 75 | Not tested - no SAM reference | | | | | | | |
| 76 | 172 | 7.54 | 14.89 | 11.23 | 46.73 | 125.33 | 0.08 | no |
| 77 | Not tested - very similar to code 76 | | | | | | | |
| 78 | Not tested - very similar to code 76 | | | | | | | |
| 79 | Not tested - no roll made | | | | | | | |
| 80 | Not tested - no SAM reference | | | | | | | |
| 81 | 87 | 5.38 | 9.53 | 13.94 | 25.19 | 106 | 8.615 | no |
| 82 | 118 | 5.52 | 10.84 | 13.72 | 30.81 | 59.4 | 1.103 | no |
| 83 | 160 | 6.12 | 13.5 | 10.85 | 35.94 | 76.17 | 0.089 | no |
| 84 | Not tested - SAM too high with respect to target | | | | | | | |
| 85 | Not tested - SAM too high with respect to target | | | | | | | |
| 86 | Not tested - SAM too high with respect to target | | | | | | | |
| 87 | Not tested - no SAM reference | | | | | | | |
| 88 | Not tested - SAM too low with respect to target | | | | | | | |
| 89 | Not tested - SAM too low with respect to target | | | | | | | |
| 90 | 120 | 5.53 | 10.75 | 14.33 | 36.94 | 73.5 | 0.516 | no |
| 91 | Not tested - no roll made | | | | | | | |
| 92 | Not tested - no SAM reference | | | | | | | |
| 93 | Not tested - SAM too low with respect to target | | | | | | | |
| 94 | Not tested - SAM too low with respect to target | | | | | | | |
| 95 | Not tested - SAM too low with respect to target | | | | | | | |
| 96 | Not tested - SAM too low with respect to target | | | | | | | |
| 97 | Not tested - SAM too low with respect to target | | | | | | | |
| 98 | Not tested - SAM too low with respect to target | | | | | | | |
| 99 | Not tested - SAM too low with respect to target | | | | | | | |
| 100 | Not tested - SAM too low with respect to target | | | | | | | |
| 101 | 142 | 5.5 | 11.7 | 13.1 | 27.8 | 53.5 | 0.15 | no |
| 102 | 133 | 5.6 | 11.9 | 14.1 | 33.1 | 67.3 | 0.12 | no |
| 103 | 129 | 6.2 | 12.3 | 16.9 | 50.9 | 87 | 0.49 | no |
| 104 | 160 | 4.8 | 12.3 | 16.8 | 35.6 | 59.3 | 0.19 | no |
| 105 | 168 | 5.4 | 12.3 | 18.1 | 27.3 | 47 | 0.13 | no |
| 106 | 138 | 5.9 | 12.4 | 15 | 45.1 | 98.8 | 0.14 | no |
| 107 | 127 | 5.4 | 11.9 | 14.2 | 35.1 | 71.4 | 0.19 | no |
| 108 | 147 | 4.5 | 10.5 | 15.8 | 29.7 | 58.4 | 0.17 | no |
| 109 | 127 | 5.5 | 10.5 | 18.3 | 34.5 | 74.8 | 0.8 | no |
| 110 | 130 | 4.6 | 12.7 | 22.8 | 48.5 | 95.5 | 0.19 | no |
| 111 | 147 | 5 | 11.6 | 17.7 | 40.6 | 76.5 | 0.14 | no |
| 112 | 172 | 4.6 | 11.9 | 16.2 | 34.8 | 68.1 | 0.18 | no |
| 113 | 165 | 4.3 | 10.8 | 17 | 40.5 | 77 | 0.16 | no |
| 114 | 122 | 4.2 | 11.8 | 26.8 | 52.7 | 119.1 | 0.23 | no |
| 115 | 163 | 6.4 | 13.2 | 14 | 47 | 89 | 0.1 | no |
| 116 | 159 | 4.7 | 11.5 | 20 | 45 | 84 | 0.14 | no |
| 117 | 195 | 5.8 | 13 | 12 | 30 | 50 | 0.2 | no |
| 118 | 188 | 5.6 | 12.8 | 18 | 42 | 75 | 0.1 | no |
| Control 1 | 175 | 7 | 17.2 | 17 | 44 | 106 | 0.1 | no |
| Control 2 | 125 | 5.5 | 12.7 | 17 | 51 | 86 | 0.1 | no |
| Control 3 | 122 | 5.7 | 15.8 | 22 | 64 | 92 | 0.09 | no |

As documented in Table 2 above, some of the codes provided a combination of benefits that was satisfactory as compared to Control 1, Control 2, and Control 3. The experimental results provided that multi-layer absorbent materials 110 including an intake layer 12 and an absorbent layer 13 that are integrated, such as through the foam forming processes described above, can provide absorbent materials that have surprisingly fast intake times for a given amount of saturation capacity, which can enable a unique combination of a thin product and/or fast intake times.

Preferable constructions of intake layers 12 and absorbent layers 13 in an integrated multi-layer absorbent material were discovered from this extensive testing. For example, it is believed that a sufficient amount of SAM should be present in the absorbent layer 13 to achieve a saturation capacity of 120 grams or more. Preferably, the absorbent layer 13 can have a SAM basis weight of at least 300 gsm, or at least 350 gsm, or at least 370 gsm, or in some embodiments, at least 400 gsm to achieve a desired saturation capacity.

Additionally, it was discovered that the intake layer 12 basis weight being too high can negatively affect rewet values, and it is believed that higher basis weight intake layers 12 may be storing too much free liquid. Preferably, the intake layer 12 has a basis weight of no more than 75 gsm, and preferably no more than 50 gsm, in order to achieve low rewet values.

It was also discovered that reducing the amount of binder fiber and/or adding a synthetic fiber (such as PET crimped fibers) in the absorbent layer 13 can help lower intake times, yet still maintain low rewet values. It is preferable to have less than about 30% binder fibers in the absorbent layer 13, and more preferably less than 15% binder fibers in the absorbent layer 13, and in some embodiments, preferably less than 10% binder fibers in the absorbent layer 13 (by weight).

Reviewing Table 2 shows that several codes provided surprisingly improved intake times while still maintaining sufficient saturation capacity and wet thickness. To this point, experimental codes provided a saturation capacity greater than 100 g, a wet thickness less than 17 mm, and a surprisingly low second intake time of less than 50 seconds. Such codes have sufficient saturation capacity and wet thickness for some intended purposes for absorbent material 110, but also provided beneficially quick second intake times. Experimental codes meeting this characterization were Code Nos. 10, 14, 15, 17, 20, 25, 26, 28, 31, 33, 36, 72, 76, 82, 83, 90, 101, 102, 104-113, and 115-118.

Some of the absorbent materials 110 were also able to be constructed in a thin manner from a perspective of both dry and wet thickness values, yet provide satisfactory rewet values, as documented in the results shown in Table 2. More specifically, experimental codes were able to provide an absorbent material 10 with a dry thickness of less than 8.0 mm, a wet thickness of less than 12.5 mm, and a rewet less than or equal to 0.14 grams. Experimental codes meeting this characterization were Code Nos. 25, 26, 28, 31, 33-36, 102, and 105.

Additionally, Table 2 also displayed that experimental absorbent materials 110 were developed to be able to have satisfactory saturation capacity and rewet values in comparison to control codes, yet provide adequately thin products from a wet thickness perspective. Specifically, some of the absorbent materials 10 were able to provide a saturation capacity greater than 125 grams, a rewet less than or equal to 0.14 grams, and a wet thickness of less than 17 mm. Experimental codes meeting this characterization were Code Nos. 14, 15, 17, 20, 26, 28, 31, 33-36, 72, 76, 83, 102, 105, 115, 118.

Further testing was conducted to determine various properties of absorbent materials with a high percentage of SAM in the absorbent layer 13. Table 3 provides various composition coding (e.g., A, B, C, etc.) and associated contents for the various absorbent materials created in Table 4. All codes in Table 4 were created as an absorbent material 10 including an intake layer 12 having a basis weight of 40 gsm formed from the respective designated content as noted in Tables 3 and 4 and that was foam formed along with the absorbent layer 13 on top of a polypropylene spunbond removable carrier sheet (having a basis weight of about 11 gsm) serving as the containment layer 17 for processing purposes, but was removed for testing properties of the absorbent material.

TABLE 3
Composition and Contents for Various Layers in Codes

| Composition | Contents |
|---|---|
| A | 25% NBSK, 40% T255 bico, 10% Barnett PET, 25% |
| | CMC535 |
| B | 25% eucalyptus, 40% T255 bico, 10% Barnett PET, |
| | 25% CMC535 |
| C | 25% NBSK, 75% T255 bico |
| D | 50% NBSK, 20% T255 bico, 30% PET |
| E | 35% T255 bico, 65% PET |
| F | 20% T255 bico, 80% PET |

TABLE 4
Various Codes Having High SAM in Absorbent Layer

| | Abs. | | | | | | | |
|---|---|---|---|---|---|---|---|---|
| | layer | Abs. | Abs. | Abs. | | | | Total |
| | SAM | layer | layer | layer | | Abs. layer | Containment | moisture |
| | (gsm, | fiber | SAM % | SAM % | Intake layer | fiber | layer | at reel |
| Code No. | ambient) | (gsm) | (target) | (actual) | composition | composition | composition | (g/g) |
| 108 | 337 | 20 | 94 | 87.2 | E | A | None | 1.40 |
| 109 | 367 | 40 | 90 | 77.1 | E | A | None | 1.85 |
| 110 | 333 | 10 | 97 | 82.5 | E | A | None | 1.74 |
| 112 | 342 | 10 | 97 | 81.0 | E | B | None | 1.66 |
| 113 | 338 | 20 | 94 | NT | E | B | None | 1.62 |
| 116 | 397 | 20 | 95 | NT | F | A | None | 1.82 |
| 201 | 397 | 20 | 95 | NT | F | A | None | 1.84 |
| 117 | 393 | 20 | 95 | NT | F | A | None | 2.61 |
| 202 | 399 | 20 | 95 | NT | F | A | None | 2.22 |
| 203 | 404 | 20 | 95 | NT | E | C | None | 2.61 |
| 204 | 436 | 20 | 96 | NT | E | C | None | 2.56 |
| 205 | 71 | 0 | 100 | NT | E | None | C, 20 gsm | 0.75 |
| 118 | 418 | 40 | 91 | NT | E | D | None | 1.96 |
| 206 | 230 | 20 | 92 | NT | E | D | None | 2.00 |
| 207 | 414 | 20 | 95 | NT | E | D | None | 2.19 |
| 208 | 453 | 40 | 92 | NT | E | D | None | 2.64 |

Experimental codes were created with target SAM values as a percentage of the total weight of the absorbent layer 13, but several exemplary codes were also tested for actual SAM weight of the absorbent layer 13 through the Sulfated Ash Test Method as described herein. As noted in Table 4, the actual SAM percentage of the absorbent layer 13 was less than the target SAM percentage in the absorbent layer 13, however, it can be seen that several of the exemplary codes had actual SAM percentages greater than 80%, greater than 81%, greater than 82%, greater than 85%, and even as high as 87.2% (by total weight of the absorbent layer 13). Of course, the disclosure is intended to cover actual SAM percentages in the absorbent layer 13 beyond these ranges, as previously discussed in this disclosure, such as greater than 90%, 91%, 92%, 93%, 94%, 95%, 96%, 97%, 98%, and 99%. For the basis weight (ambient) of the SAM in the absorbent layer, bone dry SAM basis weight was first measured by subtracting the fiber-only basis weight from the total basis weight. Then the “ambient” SAM basis weight was calculated by multiplying the bone dry SAM basis weight by 1.1 (assuming 10% moisture pick-up at ambient conditions).

Importantly, all of the experimental codes from Table 4 demonstrated relatively low SAM swell in the process of foam forming the codes. For example, all codes had less than 3 grams of water per gram of dry composite material when rolled up at the reel. This property of grams of water per gram of dry composite material was measured by cutting a 5″×10″ sample from the material at the reel when the material had not been fully dried, and weighed in an as is wet condition and then compared to the dry weight after the sample is sufficiently dried in a high speed drier or convection oven, such that the moisture of the absorbent material at the reel=(wet sample weight−dry sample weight)/dry sample weight. Not to be bound by theory, but it is believed that having low SAM swell (or low moisture in the SAM) after processing of the SAM in the foam forming mixture is important in preventing the thickness of the absorbent layer 13 from increasing and reducing the fiber network connectivity, if fibers are present in the absorbent layer 13. If the SAM swell is too great, the fiber network can be disrupted sufficiently such that the SAM can no longer be considered bound within the fiber matrix of the absorbent layer 13.

Some of the exemplary codes described in Table 4 above were tested for saturation capacity (Sat. Cap.) under the Saturation Capacity Test, first, second, and third intake times under the FIUP Test, and rewet under the Rewet Test. Experimental codes were also measured for dry and wet thickness as well.

TABLE 5
Sat. Cap., Intake Times, Dry and Wet Thicknesses, and Rewet for Selected Codes

| | Sat. Cap | Dry thick | Wet thick | 1st | 2nd | 3rd | Rewet |
|---|---|---|---|---|---|---|---|
| Code No. | (g) | (mm) | (mm) | intake (s) | intake (s) | intake (s) | (g) |
| 108 | 147 | 4.5 | 11 | 16 | 30 | 58 | 0.17 |
| 109 | 100 | 4.5 | 10 | 21 | 47 | 123 | 2.01 |
| 110 | 130 | 4.6 | 13 | 23 | 49 | 96 | 0.19 |
| 112 | 172 | 4.6 | 12 | 16 | 35 | 68 | 0.18 |
| 113 | 165 | 4.3 | 11 | 17 | 41 | 77 | 0.16 |
| 116 | 159 | 4.7 | 12 | 20 | 45 | 84 | 0.14 |
| 201 | NT | NT | NT | NT | NT | NT | NT |
| 117 | 195 | 5.8 | 13 | 12 | 30 | 50 | 0.2 |
| 202 | NT | NT | NT | NT | NT | NT | NT |
| 203 | NT | NT | NT | NT | NT | NT | NT |
| 204 | NT | NT | NT | NT | NT | NT | NT |
| 205 | NT | NT | NT | NT | NT | NT | NT |
| 118 | 188 | 5.6 | 13 | 18 | 42 | 75 | 0.1 |
| 206 | NT | NT | NT | NT | NT | NT | NT |
| 207 | NT | NT | NT | NT | NT | NT | NT |
| 208 | NT | NT | NT | NT | NT | NT | NT |
| Control 1 | 185 | 6.4 | 17 | 15 | 58 | 120 | 0.2 |
| Control 2 | 125 | 6.4 | 13 | 16 | 41 | 70 | 0.17 |

As demonstrated in Table 5, selected codes that were foam formed with high SAM percentages in the absorbent layer 13 had comparatively lower dry thicknesses and at least parity, if not thinner wet thicknesses compared to Control Codes, yet the intake times of the exemplary codes had quicker second and third intake times compared to Control Codes, while providing first intake times and rewet and saturation capacity values substantially at parity to Control Codes. One exception to this was code no. 109, which had a rewet of 2.01 g, which is substantially higher than the Control Codes. It is believed that code no. 109 did not have sufficient capacity in the absorbent layer 13.

Additional testing was completed on select experimental codes described above to test for dry thickness, wet thickness, first intake, second intake, and third intake in the Modified FIUP Test that provided a different surfactant on a 12 gsm topsheet, as described further in the Test Methods section herein. The results of the Modified FIUP Test are documented in Table 6, with the exception that saturation capacity being documented in Table 6 being from results of the FIUP Test.

TABLE 6
Intake, Dry & Wet Thicknesses, and Rewet for
Selected Codes under the Modified FIUP Test

| | Sat. | Dry thick | Wet thick | 1st | 2nd | 3rd | Rewet |
|---|---|---|---|---|---|---|---|
| Code No. | Cap* (g) | (mm) | (mm) | intake (s) | intake (s) | intake (s) | (g) |
| 14 | 163 | 6.8 | 13.0 | 10.9 | 43.4 | 87.4 | 0.10 |
| 20 | 173 | 7.2 | 13.3 | 13.1 | 52.2 | 108.1 | 0.10 |
| 26 | 176 | 5.3 | 11.8 | 17.0 | 45.8 | 74.5 | 0.09 |
| 28 | 138 | 6.1 | 11.2 | 16.7 | 56.3 | 122.3 | 0.73 |
| 31 | 185 | 4.7 | 11.5 | 18.0 | 45.7 | 86.2 | 0.12 |
| 36 | 135 | 5.3 | 10.4 | 13.6 | 43.1 | 84.7 | 0.13 |
| 101 | 142 | 4.8 | 11.7 | 17.5 | 42.5 | 76.4 | 0.12 |
| 102 | 133 | 5.7 | 12.7 | 15.4 | 55.8 | 109.0 | 0.09 |
| 103 | 129 | 5.9 | 11.6 | 15.8 | 56.1 | 88.2 | 1.08 |
| 106 | 138 | 5.4 | 12.0 | 21.0 | 53.5 | 93.8 | 0.15 |
| 108 | 147 | 4.6 | 11.6 | 18.0 | 47.3 | 101.4 | 0.09 |
| 113 | 165 | 5.1 | 10.6 | 19.2 | 49.3 | 112.2 | 0.13 |

As documented in Table 6, the Modified FIUP Test results also demonstrated that several codes provided a unique combination of benefits. The experimental results in Table 6 continued to demonstrate that multi-layer absorbent materials 110 including an intake layer 12 and an absorbent layer 13 that are integrated, such as through the foam forming processes described above, can provide absorbent materials that have surprisingly fast intake times for a given amount of saturation capacity, which can enable a unique combination of a thin product and/or fast intake times.

The Modified FIUP Test results are consistent with the FIUP test results documented in Table 2 that demonstrated that limiting the intake layer 12 basis weight can help keep rewet values lower, and as such, it may be preferable in some codes to have an intake layer 12 with a basis weight of no more than 75 gsm, and preferably no more than 50 gsm, in order to achieve low rewet values.

The Modified FIUP Test results also demonstrate consistent results in that reducing the amount of binder fiber and/or adding a synthetic fiber (such as PET crimped fibers) in the absorbent layer 13 can help lower intake times, yet still maintain low rewet values. It is preferable to have less than about 30% binder fibers in the absorbent layer 13, and more preferably less than 15% binder fibers in the absorbent layer 13, and in some embodiments, preferably less than 10% binder fibers in the absorbent layer 13 (by total weight of the absorbent layer 13).

Reviewing Table 6 shows that several codes provided surprisingly improved intake times while still maintaining sufficient saturation capacity and wet thickness. To this point, many of the selected experimental codes within Table 6 provided a wet thickness less than 17 mm, and a surprisingly low second intake time of less than 50 seconds. Such codes have sufficient saturation capacity and wet thickness for some intended purposes for absorbent material 110, but also provided beneficially quick second intake times. Experimental codes meeting this characterization in the Modified FIUP Test from Table 6 were Code Nos. 14, 26, 31, 36, 101, 108, and 113. All of these experimental codes also had saturation capacities greater than 125 g.

Some of the absorbent materials 110 were also able to be constructed in a thin manner from a perspective of both dry and wet thickness values, yet provide satisfactory rewet values, as documented in the results shown in Table 6. More specifically, experimental codes were able to provide an absorbent material 10 with a dry thickness of less than 8.0 mm, a wet thickness of less than 12.5 mm, and a rewet less than or equal to 0.14 grams. Experimental codes meeting this characterization from the selected codes in the Modified FIUP Testing shown in Table 6 were Code Nos. 26, 31, 36, 101, 108, and 113.

Additionally, Table 6 also displayed that experimental absorbent materials 110 were developed to be able to have satisfactory saturation capacity and rewet values in comparison to control codes, yet provide adequately thin products from a wet thickness perspective. Specifically, some of the absorbent materials 10 were able to provide a saturation capacity greater than 125 grams, a rewet less than or equal to 0.14 grams, and a wet thickness of less than 17 mm. Experimental codes meeting this characterization were Code Nos. 14, 20, 26, 31, 36, 101, 102, 108, and 113.

Horizontal Side Compression Testing was also conducted for select codes having high percentage of SAM in the absorbent layer 13, the results of which are documented in Table 7.

TABLE 7
Horizontal Side Compression Testing Results for Selected Codes

| | Cycle 1 | Cycle 10 |
|---|---|---|
| | energy | Recovery |
| Code | (g*cm) | (%) |
| 108 | 606 | 96 |
| 109 | 488 | 94 |
| 110 | 390 | 95 |
| 112 | 749 | 99 |
| 113 | 473 | 97 |
| Control 1 | 1000 | 90 |
| Control 2 | 1100 | 92 |

Selected codes as shown in Table 7 that were foam formed with high SAM percentages in the absorbent layer 13 demonstrated important benefits over Control Codes from the Horizontal Side Compression Testing Results. Table 7 documents that the selected codes provided significantly lower cycle 1 energy than the Control Codes, as well as the Cycle 10 recovery and resiliency for the selected codes being improved over the Control Codes meaning that the selected codes with greater than 80% SAM in the absorbent layer 13 provide very flexible absorbent materials 10, 110, 210. Enhanced flexibility of absorbent materials 10, 110, 210 used for personal care absorbent articles can provide enhanced comfort for a user and can also help lead to reduced leakage by providing a better fit for such personal care absorbent articles.

Preferably, absorbent materials 10, 110, 210 can include a cycle 1 energy of less than 1000 g*cm, or more preferably, less than 950, 900, 850, 800, 750, 700, 650, 600, 550, or even 500 g*cm. Absorbent materials 10, 110, 210 can also include a cycle 10 recovery of greater than 92%, or more preferably, greater than 93%, 94%, 95%, 96%, 97%, or 98%.

Testing was also conducted on select codes having high percentages of SAM in the absorbent layer 13 to determine the integrity of such absorbent materials. This testing conducted was pursuant to the Internal Cohesion Testing and the Shake Testing, as described in the Test Methods section herein.

The results of the Internal Cohesion Testing for select codes having high percentage of SAM in the absorbent layer 13 are documented in Table 8. Control Code 2 was not tested in the Internal Cohesion Testing (marked NT).

TABLE 8
Internal Cohesion Testing Results for Selected Codes

| Code | Dry (kg) | Wet (kg) |
|---|---|---|
| 108 | 1.4 | 1.0 |
| 109 | 1.23 | 1.05 |
| 110 | 0.8 | 0.5 |
| 112 | 0.9 | 0.8 |
| 113 | 1.1 | 0.9 |
| Control 1 | 0.4 | 0.5 |
| Control 2 | NT | NT |

Selected codes with high amounts of SAM surprisingly provided higher dry and wet values in the Internal Cohesion Testing. These results were unexpected due to the fact that absorbent materials having significant amounts of SAM in their absorbent layer 13 were not believed to be able to provide high cohesion values, especially in the absence of internal adhesives or adhesives attaching such absorbent layers 13 to other layers of the absorbent material 10, 110, 210, such as the intake layer 12 and/or the containment layer 17. Preferred embodiments of the absorbent material 10, 110, 210 can include an Internal Cohesion Test dry value greater than 0.4, more preferably greater than 0.5, 0.6, 0.7, 0.8, 0.9, 1.0, 1.1, 1.2, or 1.3. Preferred embodiments of the absorbent material 10 can include an Internal Cohesion Test wet value greater than 0.5, more preferably greater than 0.6, 0.7, 0.8, 0.9, or 1.0.

Shake Testing, as described in the Test Methods section herein, was also performed on selected codes of absorbent materials 10 having greater than 80% SAM in the absorbent layer 13. The results of the Shake Test testing are documented in Table 9. The Control Codes were not tested in the Shake Test Results.

TABLE 9
Shake Test Results for Selected Codes

| | Average # of shakes |
|---|---|
| Code | before break |
| 108 | 22.5 |
| 109 | NT |
| 110 | 2.25 |
| 112 | 5 |
| 113 | 8 |

As documented in Table 9, preferable codes of absorbent materials had an average number of shakes before break of at least 2 in the Shake Test. The results of the Shake Test were unexpected in that the absorbent materials 10, 110, 210 having absorbent layers 13 including significant amounts of SAM in their absorbent layer 13 (such as greater than 80%) were expected to break down easily, especially in the absence of internal adhesives or adhesives attaching such absorbent layers 13 to other layers of the absorbent material 10, 110, 210 such as the intake layer 12 and/or the containment layer 17 as more traditional absorbent materials are formed. Preferable embodiments of absorbent materials 10, 110, 210 can provide an average number of shakes to break of at least 2, or more preferably at least 3, 4, 5, 6, 7, 8, 9, 10, 11, 12, 13, 14, 15, 16, 17, 18, 19, 20, 21, or 22.

As documented from testing above, it was unexpectedly discovered that absorbent materials 10, 110, 210 can be made in a foam forming process with an absorbent layer 13 having greater than 80% SAM in the absorbent layer 13, yet still provide sufficient structural integrity for processing and handling as well as beneficial performance characteristics. Not to be bound by theory, but it is believed that the mixing of some fibers of the intake layer 12 with the SAM and/or fibers of the absorbent layer 13 due to the foam forming process may provide structural integrity to the absorbent material 10, 110, 210 even with high SAM content in the absorbent layer 13. As described above, in some preferable embodiments, the absorbent layer 13 can include a plurality of fibers in the absorbent layer 13 in addition to the SAM that may also help provide improved integrity for the absorbent layer 13, and thus, the overall absorbent material 10, 110, 210. In some embodiments, it is preferable to have at least 15%, or more preferably, at least 20% of the fibers of the absorbent layer 13 be absorbent fibers (by weight of the fibers of the absorbent layer 13). In some embodiments, it is preferable to have at least 15%, or at least 20%, or at least 25%, or at least 30%, or at least 35% or more by weight of the fibers of the absorbent layer 13 be binder fibers (by weight of the fibers of the absorbent layer 13). Providing some amount of binder fibers in the absorbent layer 13 can provide integrity to the overall structure of the absorbent material 10, 110, 210. In some embodiments, the absorbent layer 13 can also include synthetic fibers that are non-absorbent, such as at least 5%, or at least 10% or more of the fibers of the absorbent layer 13 can be synthetic fibers (by weight of the fibers of the absorbent layer 13). In some embodiments, the synthetic fibers in the absorbent layer 13 are preferably at least 4.0 mm in length to provide additional integrity to the absorbent layer 13.

### Test Methods

### Saturation Capacity Test:

The saturation capacity of experimental codes was measured according to the following protocol: specimens were prepared to following dimensions: 220 mm in length and 67 mm in width. The specimens were sealed in a spunbond nonwoven bag prior to the test to prevent material loss due to SAM swelling during the test. The saturation capacity test was performed using a table top saturation capacity tester as described herein. First, the dry sample mass is measured. Then, the samples were saturated for 20 minutes in a saline solution (0.9 wt % NaCl) and then allowed to drip dry for 1 minute. The samples were next placed on the mesh screen of the table top saturation capacity tester having 0.25 inch (6.4 mm) openings (commercially available from Taconic Plastics Inc. Petersburg, N.Y.) which, in turn, is placed on a vacuum box and covered with a flexible rubber dam material, such as a latex sheet. A vacuum of 3.5 kilopascals (0.5 pounds per square inch) is drawn in the vacuum box for a period of 5 minutes. The sample is then removed from the vacuum box and weighed against to determine a saturated, or wet weight of the sample. If material, such as superabsorbent material or fiber, is drawn through the fiberglass screen while on the vacuum box, a screen having smaller openings should be used. Alternatively, a piece of the tea bag material (such as heat sealable tea bag material (grade 542, commercially available from the Kimberly-Clark Corporation)) can be placed between the material and the screen and the final value adjusted for the fluid retained by the tea bag material. The saturation capacity is the total weight of the wet sample minus the sample dry weight.

### FIUP Test:

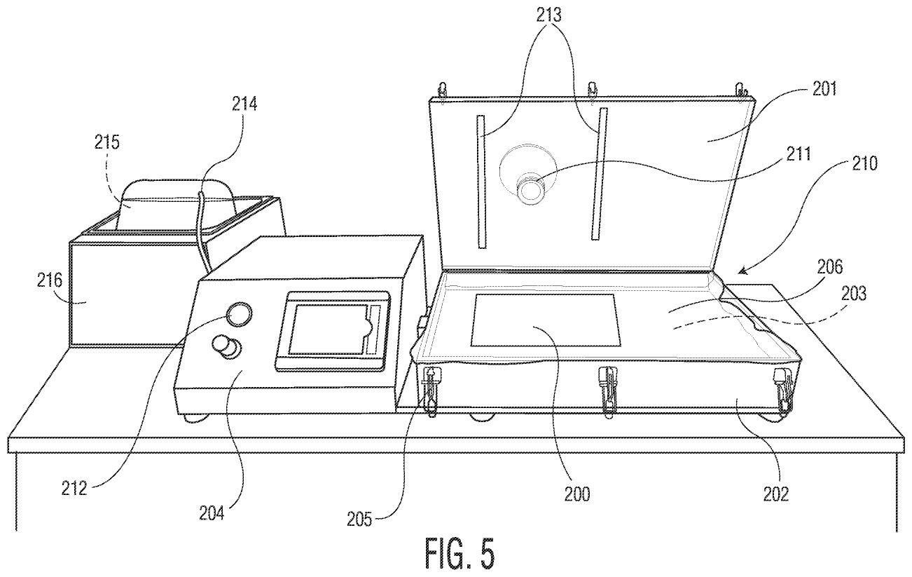

The first, second, and third intake times of experimental codes were measured according to the following protocol and by the exemplary equipment illustrated in FIG. 5 for a Fluid Intake Under Pressure (FIUP) Test. The specimens 200 were prepared to the following dimensions: 215 mm in length and 62 mm in width and placed between the topsheet with flaps and the back sheet from the commercially available Poise® Ultra Thin Moderate 4-drop Regular pads. The topsheet can be a 20 gsm polypropylene spunbond nonwoven liner material with hydrophilic treatment, such as XHBY21520/YSQS215 material provided by Lanxi Xinghan Plastic Material Co. (Hengyao). The back sheet can be a 24 gsm polyethylene film. For specimens without an intake layer, a fresh piece of 185 mm by 49 mm intake layer material of 42 gsm polyethylene/polypropylene bicomponent TABCW (JingLan) was placed over the core to serve as an intake layer and had 6 gsm of adhesive applied (from a swirl of adhesive on release paper) to the top and bottom of the intake layer. The sides of the sample 200 are sealed with double-sided tape. The samples 200 were brought to TAPPI conditions for at least 4 hours.

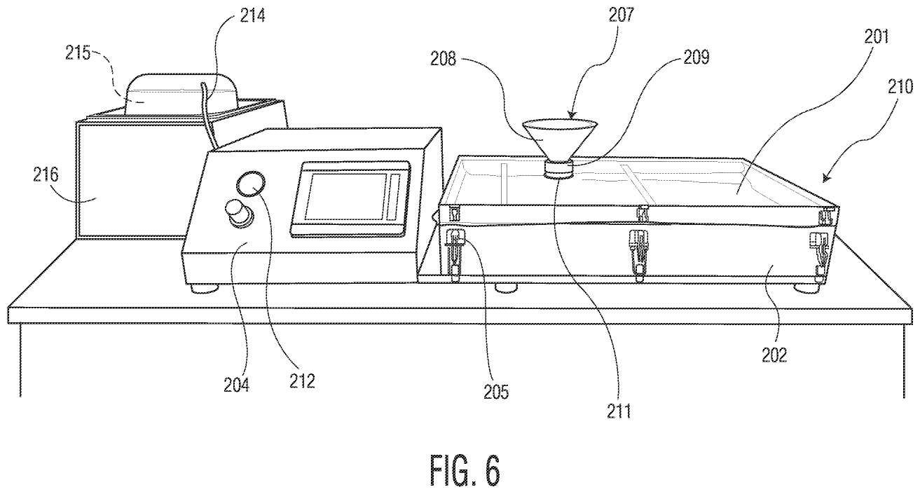

The FIUP Test uses a “bladder box” 210 as illustrated in FIGS. 5 and 6. The bladder box 210 includes a cover 201, a housing 202, an inflatable bladder 203, and a control unit 204. The cover 201 can be made from a clear material, such as clear, cast acrylic. The cover 201 can be hinged to the housing 202. The housing 202 can be constructed from aluminum can be of the size of 62 cm×40 cm×15 cm. The housing 202 can also include latches 205, such as the three latches 205 depicted in FIGS. 5 and 6, for securing the cover 201 to the housing 202. When the cover 201 is opened, the test specimen 200 can be laid on top of a thin plastic film 206 laid on top of the bladder 203. The test specimen should be laid on the film 206 and bladder 203 such that the specimen 200 is centered under the intake port 207. The bladder 203 can be an inflatable bladder, such as a Aero Tec Labs bladder, that can fit within the housing 202 and that can be filled with compressed air.

The intake port 207 can include a threaded funnel 208 that threads into a threaded plug 209 having a 1″ diameter opening at the bottom of the threaded plug 209 and provides for communication to the test specimen 200. The intake port 207 can also include an O-ring 211 that seals the threaded plug 209 with the cover 201. The intake port 207 can also include a round, flat gasket (not shown) to seal between the threaded funnel 208 and the threaded plug 209. The bottom of the intake port 207 should be flush with the underside of the cover 201.

The control unit 204 can be a process controller such as 1/16 DIN Fuzzy Logic; Example: Omega, part number CN48001-F1-AL2:G1, or equivalent, and can be configured to be in communication with a pressure transmitter measuring the pressure of the bladder 203. An exemplary pressure transmitter can be a Omega Engineering, part number PX181-015GSV. The control unit 204 can also be in communication with a fluid dispensing pump (e.g., Cole-Parmer peristaltic pump, P/N 07551-20) and pump head (P/N 77201-60) that is set up to deliver fluid to the test sample at a specified flow rate of 8 mL/s via clear pump tubing 214 (e.g., Masterflex clear tubing L/S 14, L/S 25, or L/S 17). The end fitting on the tubing can have an exit diameter of 0.125″, such as Cole-parmer Reducing Connector, Nylon, ¼″× 3/16″, Item No. 30622-30.

After the test specimen 200 is set in the bladder box housing 202 by being centered below the intake port 207. As illustrated in FIG. 5, the bottom of the cover 201 can include two strips of hook tape 213 (e.g., Item #1055, Dariss Brand) that are used to help secure the test specimen 200. After the sample is centered, the cover 201 is closed and latches 205 are latched. The hook tape 213 should be applied to the cover 201 such that the hook tape 213 only touches non-absorbent material of the test specimen 200. The power for the control unit 204 is turned on to set the bladder 203 pressure to 0.25 psi. Once the control unit 204 identifies that the bladder 203 has reached a stable pressure of 0.25 psi, a pressure gauge 212 can be checked to verify that the pressure in the bladder 203 is within 0.25+/−0.01 psi. If the pressure is not within 0.01 psi of 0.25 psi, the test should be stopped and the set pressure should be adjusted to compensate until the pressure gauge 212 reads within 0.01 psi of 25 psi.

The insult liquid used for the FIUP test is 0.9±0.005% (w/w) aqueous isotonic saline 215 that is placed in a heated water bath 216 at a temperature of 98.6±1.8° F./37±1° C. prior to testing. The saline solution 215 temperature should be confirmed with a thermometer prior to insulting the test specimen 200. The first insult is a 25 mL insult and is supplied through the intake port 207 by aiming the fluid at the bottom angled side of the funnel 208. The first intake time of the first insult begins once the pump is turned on to deliver fluid to the intake port 207 and continues until all droplets of fluid have been absorbed within top layer of the test specimen 200. The second 25 mL insult is applied 15 minutes after the first insult is fully absorbed and the second intake time is measured in the same manner as the first insult time. The third 25 mL insult is applied 15 minutes after the second insult is fully absorbed and the third intake time is measured in the same manner as described above.

After the third intake time is recorded, a timer should be started to allow two minutes to pass. The control unit 204 is then calibrated to stop the test by releasing the bladder 203 pressure in the bladder box 210.

If any point during the insult testing there is any fluid runoff beyond the test specimen 200 on to the plastic sheet 206 covering the bladder 203, the test should be marked as a “FAIL” and not recorded.

The testing is conducted with a sample set of N=5.

### Modified FIUP Test:

The Modified Fluid Intake Under Pressure Test (referred to as the “Modified FIUP Test”) is performed the same as the FIUP Test described above and as illustrated in FIGS. 5 and 6, with the following exceptions for sample 200 preparation.

An exemplary absorbent material is cut to 215 mm in length and 62 mm in width. A topsheet of a 12 gsm polypropylene spunbond liner is cut to four inches by ten inches area and hand sprayed with 1.0% surfactant solution of sodium dodecyl sulfide (SDS) using a Preval sprayer. The solution add-on is measured gravimetrically when wet prior to air-drying the sample and should be provided such that the surfactant add-on is 0.27% (by mass of liner), with a standard deviation of 0.06%. Any topsheets outside of this add-on range of surfactant should not be used. Flaps are removed from a Poise® Ultra Thin Moderate 4-drop Regular pad and a back sheet of a 24 gsm polyethylene film is also prepared. The exemplary absorbent material is placed between the surfactant treated topsheet and the 24 gsm PE back sheet and a spiral-pattern 6 gsm sheet adhesive is applied on the top and bottom side of the absorbent material to adhere to the topsheet and back sheet, respectively. The flaps are applied to the spunbond topsheet with double sided tape adhesive. For specimens without an intake layer, a fresh piece of 185 mm by 49 mm intake layer material of 42 gsm polyethylene/polypropylene bicomponent TABCW (JingLan) was placed over the absorbent material core to serve as an intake layer and had 6 gsm of adhesive applied (from a swirl of adhesive on release paper) to the top and bottom of the intake layer. The sides of the sample 200 are sealed with double-sided tape. The samples 200 were brought to TAPPI conditions for at least 4 hours.

### Rewet Test:

The rewet for the experimental codes was measured by using the same specimen from the FIUP test discussed above, and can also be conducted after the Modified FIUP test as described above. The rewet test is a continued test after the FIUP test (or Modified FIUP Test) was completed. Specifically, 2 minutes after the third insult of the FIUP test is complete, the sample is removed from the bladder box 210 and placed onto a flat surface, insult side facing up. The test is completed using two stacked pieces of blotting paper (e.g., 300 g/m2 (100 lb./ream)—Verigood Grade 88 by 300±13 mm (3.5 by 12±0.5 inch)) to absorb the free saline from the insulting point of the specimen 200 under an external load after the FIUP test. The two pieces of blotting paper were pre-weighed and each had a dimension of 3.5″×12″ and would be placed to cover the center of the specimen's insulting point by removing the FIUP testing board and adding a cylindrical weight of 249 g and having a 1 inch diameter on the top of the blotting papers at the insult point to create a pressure of 0.7 psi for a period of two minutes. The mass of the wetted blotter papers is then measured and the rewet is calculated as: Rewet=total wet mass−dry mass. The higher the amount of wet weight measured from the test, the higher the rewet value the specimen had.

### Thickness Measurements:

Both dry thickness and wet thickness measurements of experimental codes were measured as part of the FIUP test discussed above, or can be measured after conducting the Modified FIUP Test discussed above. The thickness measurements utilize a standard bulk tester with clear, cast acrylic foot that provides 0.05 psi. The dry thickness measures the dry bulk at the center point when the sample is dry and measures the thickness of the sample in a full product form as placed in a Poise® Ultrathin chassis that includes flaps, outer cover, and liner (only the outer cover and liner form part of the thickness measurement, as the flaps are outside the platen area). The wet thickness is measured after the rewet testing is complete by measuring the bulk at the center point.

### Sulfated Ash Test Method:

The Sulfated Ash Test Method is used to calculate the percentage of SAM in an absorbent material 10, or in a particular layer, such as the absorbent layer 13, of an absorbent material 10. The test method converts the sodium or other cations in carboxyl salt polymers, such as Polyacrylate or Carboxymethyl Cellulose SAM, to the corresponding sulfate salt. The sulfate salt is determined gravimetrically and is calculated to the weight of the carboxyl salt polymer by applying a standard factor determined from a sample of the pure polymer. The sample is charred over a low flame or in a muffle furnace to remove the bulk of the volatile matter, cooled, moistened with a 1:1 sulfuric acid:water solution, the excess acid volatilized, and the ashing completed as in a regular ash determination.

The method can be applied to a wide range of sample sizes, but for purposes herein, was used to determine the percentage of SAM within the absorbent layer 13 of the absorbent material 10. The presence of any other inorganic compound or cation will give a positive interference. Accuracy is governed by the degree to which interferences can be corrected for and by how accurately the standard factor is known.

A standard factor was calculated for this testing based on a sample of pure SAM (Evonik 5660) and resulted in a standard factor of 1.98 from the formula of Standard Factor (F)=grams of polymer/grams of sulfated ash.

Three samples of each code are tested for percentage of SAM and then averaged. Each sample is to be cut to a size of 215 mm×62 mm. The samples should be in the range of 1-10 g, with most likely being in the 4-7 g range. Each sample is calculated for SAM percentage by placing the sample in a fired and tared crucible, fired in a muffle furnace at 600° C. until most of the carbonaceous materials have burned off. This and the next ignition step are completed in an exhaust hood to remove smoke and vapors. Next, the sample is cooled and a 1:1 sulfuric acid:water solution (by volume) was added. In preparing the 1:1 sulfuric acid solution, add the sulfuric acid to the water very slowly and mix slowly. A heat resistant container should be used for the mixing vessel due to heat created in the mixing of the solution. Proper PPE should be worn and the mixing should occur in a sink or other secondary containment vessel.

After the sulfuric acid solution is added to the sample, the solution is allowed to fume off. The solution can be allowed to slowly evaporate any excess acid over a low flame or on a hot plate to avoid spattering. Further ignition for the sample is then conducted by placing the sample in a muffle at 800° C. for sixty (60) minutes or until the ash is free of carbon.

The crucible is then cooled in a desiccator and weighed. The SAM estimate was calculated from the sulfated ash through the formula Percent SAM=(A×F)/C; wherein (A) is the weight of sulfated ash from the sample, (F) is equal to the standard factor (1.98 for testing conducted herein), and C is the weight of sample being analyzed.

Horizontal Side Compression Test: The Horizontal Side Compression Test compresses the absorbent material 10 horizontally.

The Test protocol has 10 cycles of dry testing. The absorbent material 10 may be tested with or without flaps depending on the purpose of investigation. Test outputs used in this description include cycle 1 energy (g*cm) and cycle 10 recovery (%).

Sample materials are placed in product form by placing the absorbent material 10 sample in a Poise® Ultrathin chassis that includes a film backing layer and a liner topsheet in a rectangular product as described in the FIUP Test described above, without any flaps.

To perform the Horizontal Side Compression Test, a Constant Rate of Elongation (CRE) type of tensile tester with data acquisition unit and data acquisition program capable of collecting data such as Instron 3343 system with Bluehill program or MTS Insight 1EL system with TestWorks 4.0 is used.

The Test is conducted by warming up the tensile tester according to the manufacturer's manual. Next, verify the appropriate load cell is in the tensile tester, which should be selected from either a 50 Newton or 100 Newton maximum, depending on the peak force value of the sample being tested, such that the majority of peak load values fall between 5-95% of the load cell's full scale value. For purposes of the samples tested herein, a 100 Newton load cell was used. In this test, both edges of the absorbent material 10 are clamped between top and bottom grips of the tensile tester with the center of the sample aligned with the center of the grips and the sample centered between the grips. Turn on the computer and follow the software menu selection. Follow the manufacturer's instructions for calibrating the load cell for the tensile tester. Verify the test conditions are as documented in Table 10.

TABLE 10
Test Conditions for Tensile Tester

| Cross Head Speed | 508 +/− 5 | mm/min |
|---|---|---|
| Gauge Length | 91 | mm |
| End Compression Distance | 30 | mm |
| Load Unit | Newton | |
| Load Limit Hi | 90 | Newton |

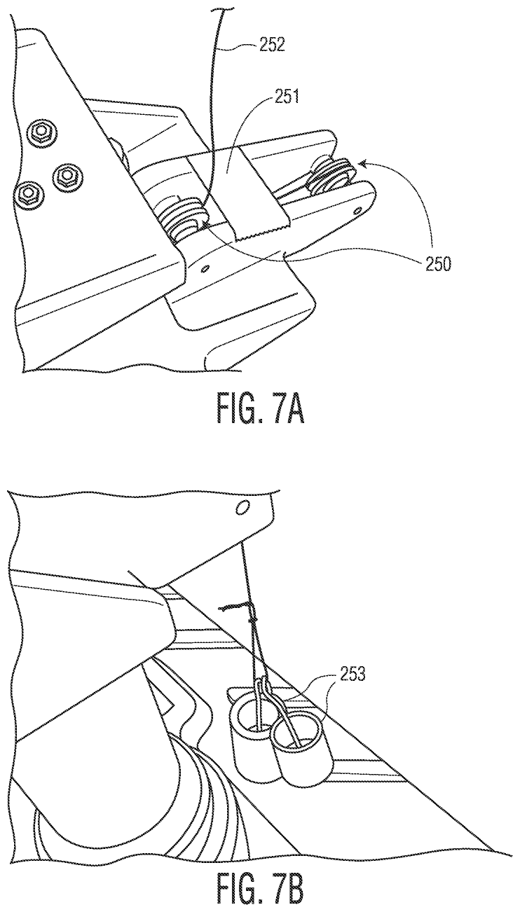

Ensure the lanyard thread is in and remains in the wheel guides 250, one in the front and two wheel guides in the back of the tester (as depicted in FIG. 7A). As depicted in FIG. 7A, a piece of masking tape 251 can be placed close to one of the back wheels 250 of the tester without touching the lanyard to prevent the lanyard 252 moving out of the wheel when the crosshead returns to its start position. Two hanging weights 253 are attached to the wheel guide at the far back of the testing unit, as depicted in FIG. 7B. Orient the weights 253 up-side down to shorten the hook length such that the weights 253 do not touch the frame.

With the lanyard attached to a hook below the load cell, adjust the crosshead so the resultant force exerted by the lanyard is less than 0.5 grams. Measure and then record the initial width of the specimen in the mid-crotch area. Then, zero the crosshead channel and start the test run. At the conclusion of the 10 cycles, measure and record the final width of the specimen in the mid-crotch area. A data report is generated that provides the cycle 1 energy (g*cm). The cycle 10 recovery % is measured as final width at cycle 10 divided by the initial width, multiplied by 100.

### Internal Cohesion Test:

The Internal Cohesion Test is used to measure the bond strength between layers of an absorbent material 10, and for purposes herein, is conducted on dry and wet absorbent materials 10 and is measured in kilograms. A Cohesion Tester, such as a Legacy Cohesion Tester can be used to perform the Test. To begin, adjust the pressure regulator to 413.69±6.89 kPa [(4.2±0.07 kg/cm²) 60±1 pound force per square inch) (psi)] by turning the regulator adjustment knob clockwise to increase the pressure, counter-clockwise to reduce the pressure.

The Touch Screen OCS Controller: After the control console is turned ON the console goes through a self-test, finishing with the Main Menu screen. Press Test to go to the Cohesion Test screen. When the number signs—#—are pressed on the Cohesion Test screen, the Numeric Keypad appears. Set the first compression time to 3.00 seconds by pressing the appropriate numbers on the number pad, then press Enter in the lower right corner. Press the Start button on the Cohesion Test screen. Ensure the test time displays the appropriate second count for the second compression time to be conducted with Dry Cohesion Testing to be set to 10.00 seconds and Wet Cohesion Testing to be set to 75.00 seconds. Then, turn on the force gauge. Ensure the Tester is configured in kg and press the peak button until the tensile at peak is displayed.

For Dry Cohesion Testing, cut a piece of 50.8 mm (2 inch) wide tape approximately 114.3 mm (4.5 inches) in length. Apply the tape to the lower specimen platform with an approximate 6 mm (0.25 inch) overlap on both the left and right sides. Cut a piece of 25.4 mm (1 inch) wide double-sided tape approximately 31 mm (1.25 in) length and apply the double-sided tape to the contact block with an approximate 3 mm (0.125 inch) overlap on two of the block's sides. Note: Do not allow the taped surfaces to come into contact with any other surface, fingers or material prior to testing. If applicable, remove the peel strip from the specimen if applicable, and without exerting pressure to the specimen, center the specimen with the body-side up on the taped lower specimen platform. Rotate the upper pressure plate until the slotted portion of the upper pressure plate is positioned at the back of the instrument and the plate locks into position. After the lower specimen platform has descended, rotate the upper pressure plate until the slotted portion of the upper pressure plate is positioned at the front of the instrument and locks into position. Hang the contact block on the hook of the force gauge, ensuring that the taped surface does not come into contact with the upper pressure plate and that the contact block hangs freely. Zero the force gauge with the contact block hanging freely. Press the TEST button on the Legacy Controller or the Start 2nd Compression on the OCS Upper Platen Rotation menu. Note: Do not zero the equipment during the 10 second testing time. OCS Controller Second Compression screen appears for the second compression and hold process. When completed, the Cohesion Test screen reappears. Record the bond strength value to the nearest 0.01 kg.

For Wet Cohesion Testing, the same method is followed with respect to the Dry Cohesion Test but the absorbent material specimen needs to be wetted. Follow the same methodology for applying the specimen to the tester described above for the Dry Cohesion Testing, but additionally cut a piece of 25.4 mm (1 inch) wide “All Purpose” tape approximately 31 mm (1.25 in) in length. Apply the “All Purpose” tape with the adhesive side out to the contact block so that it covers all the double-sided tape as noted above. Center the specimen with the body-side up on the taped lower specimen platform. Rotate the upper pressure plate until the slotted portion of the upper pressure plate is positioned at the back of the instrument. The upper pressure plate will lock into position. Then, press the Start button. Note: Do not zero the equipment during the 75 second testing time. Immediately after the first 10 seconds, dispense 1.5 mL of distilled or deionized water by positioning the nozzle tip of the dispenser on either the left or right side of the contact block in the approximate center of the end of the block. Immediately re-position the nozzle tip and dispense 1.5 mL water in the approximate center of the end of the contact block on the opposite side. Allow no more than 5 seconds to dispense the 3.0 mL of distilled or deionized water. Ensure the distilled or deionized water does not overflow the lower specimen platform, while allowing the water to soak into the specimen for the remaining 60 seconds of the testing time. When the 75 second testing time has elapsed, the lower specimen platform will descend. If the specimen releases from the tape of either the lower specimen platform or the contact block, discard the result and retest with a new specimen. If the retest results in the same specimen release, document that specimen release occurred. The application of a fresh supply of tape to the lower specimen platform and the contact block may prevent specimen release from recurring. Record the bond strength value to the nearest 0.01 kg.

### Shake Test:

The Shake Test can help detect the overall pad integrity (i.e., the ability of the absorbent layer 13 to stay in place upon insult and movement). The Shake Test is based on a test method delivered by adhesive suppliers to understand durability of pad integrity adhesive (often referred to as PIA) to hold the pad structure in place during use.

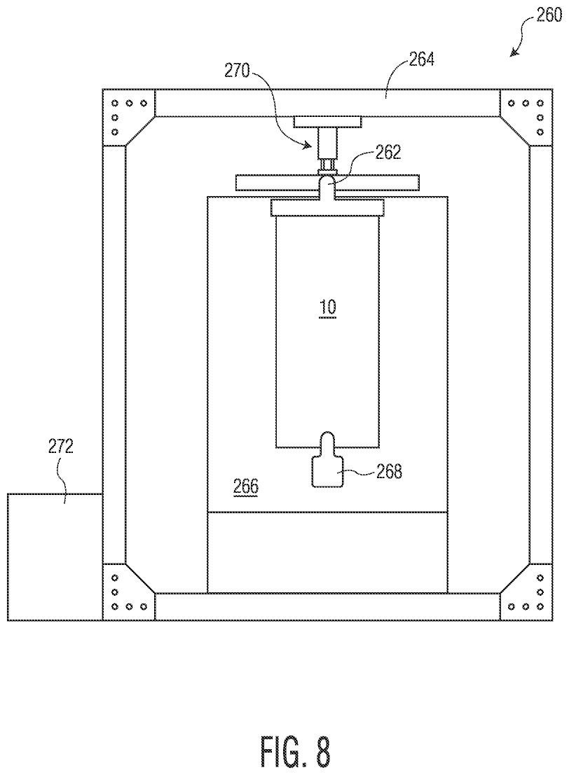

As depicted in FIG. 8, the Shake Test module 260 includes a clip 262 to hold the absorbent material 10 and a frame 264. The clip 262 holds the absorbent material 10 from the top of the absorbent material 10. A light box 266 is placed behind the absorbent material 10 to illuminate the absorbent material 10 to sufficiently see the structure of the absorbent layer 13 of the absorbent material 10. A 250 g weight clip 268 is used to attached to the absorbent material 10 sample being tested. A Pad Integrity Shaker module 270 is pneumatically connected to a source of compressed air 272. The module 270 has two output hoses (not shown) that connect to the source of compressed air 272, which can act to lower the module 270 a distance of one inch at a rate of approximately 20 inches/second and come to an abrupt stop in a lowered position. Compressed air then raises the module 270 at approximately 3 inches/second and comes to an abrupt stop in the raised position. As a result, the module 270 acts as a double-acting piston to lower and lift the clip 262 and the absorbent material 10 connected to the clip 262 to test the integrity of the absorbent material 10 sample. The module 270 is configured to have approximately a one second delay between starting the falling action to starting the rising action, as well as a one second delay between starting the rising action to starting the falling action. Because the rising of the module 270 is slower compared to the lowering of the module 270, the module 270 stays in the raised position for a shorter period of time.

Three samples of each code are prepared by cutting to a sample size of 215 mm×62 mm. A gallon of unheated 0.9% blue colored saline is prepared. Three beakers are prepared capable of holding 100 mL of the saline. Prior to hanging the absorbent material 10 a target location of 1.8 cm from the center of the absorbent material is marked on the absorbent material 10. Using two-sided tape, adhere the absorbent material 10 specimen to a bench top with the intake layer 12 side facing up. Center a 6″ high by 2″ diameter lexan tube (approximately ⅛″ thick wall, internal diameter of 1.75″ internal diameter) over the target location mark, and insert a plastic funnel to the lexan tube. Pour the 1^{st} loading of 20 mL in the funnel into the tube. The funnel spout should be angled toward the wall of the tube so that the saline flows down the side of the tube before contacting the surface of the absorbent material 10. Remove the tube and funnel from the product until the next loading. Start a timer for 5 minutes and wait. After the first 5 minute wait, then center the tube with the funnel over the target location against and pour the second loading of 20 mL into the funnel in the tube. Remove the tube and funnel from the product until the next loading. Start a second 5 minute timer and wait. After the second 5 minute wait, then center the tube with funnel over the target location mark and pour the 3rd (final) loading of 20 mL into the funnel in the tube. Remove tube and funnel. Start a third 5 minute timer and wait.

After the third and final 5 minute wait, remove the absorbent material 10 from bench top and attach the front edge of the absorbent material to clip 262 connected to the top center of the Pad Integrity Shaker module 270 with the top side of the absorbent material 10 (e.g, intake layer 12, if present) facing the user. Attach the 250 g weight clip 268 at the bottom edge of the absorbent material 10.

Press the Start button on the controller to begin the lowering the absorbent material 10 sample and begin counting each shake up to 25. A single “shake” is counted when the sample goes down. Allow the test to continue until the absorbent material 10 breaks. Record the number of shakes that caused the absorbent material 10 sample to fully break. When a “full break” occurs, stop the shaker by pressing the RESET button and record the number of shakes. The average number of shakes is calculated after completing this test for the three samples per code.

### Embodiments

Embodiment 1: An absorbent material comprising: an intake layer; and an absorbent layer; wherein the absorbent material comprises a saturation capacity greater than 125 grams, a second intake time of less than 50 seconds and a wet thickness of less than 17 mm according to the Modified Fluid Intake Under Pressure Test as described herein.
Embodiment 2: The absorbent material of embodiment 1, wherein the intake layer and the absorbent layer provide an integrated material including an interface between the intake layer and the absorbent layer, the interface including at least some fibers of the intake layer mixed with at least some fibers of the absorbent layer.
Embodiment 3: The absorbent material of embodiment 1 or 2, wherein the saturation capacity is greater than 150 grams.
Embodiment 4: The absorbent material of any one of embodiments 1-3, further comprising a third intake time of less than 85 seconds.
Embodiment 5: The absorbent material of any one of embodiments 1-4, wherein the wet thickness is less than or equal to 14.0 mm.
Embodiment 6: The absorbent material of any one of embodiments 1-5, further comprising a dry thickness less than 8.0 mm.
Embodiment 7: The absorbent material of any one of embodiments 1-6, further comprising a rewet less than or equal to 0.14 grams.
Embodiment 8: The absorbent material of any one of embodiments 1-7, wherein the intake layer comprises synthetic fibers and binder fibers.
Embodiment 9: The absorbent material of embodiment 8, wherein the binder fibers comprise between 15% to 50% of the intake layer (by total weight of the intake layer).
Embodiment 10: The absorbent material of any one of embodiments 1-9, wherein the absorbent layer comprises absorbent fibers, binder fibers, and superabsorbent material, and wherein the binder fibers comprise less than 30% of the absorbent layer (by total weight of the intake layer).
Embodiment 11: The absorbent material of any one of embodiments 1-7 or 10, wherein the intake layer comprises synthetic fibers and binder fibers, the binder fibers of the intake layer comprise less than 50% of the intake layer (by total weight of the intake layer); and wherein the absorbent layer comprises absorbent fibers, binder fibers, and superabsorbent material, wherein the binder fibers of the absorbent layer comprise less than 20% of the absorbent layer (by total weight of the absorbent layer).
Embodiment 12: An absorbent material comprising: an intake layer; and an absorbent layer; wherein the absorbent material comprises a dry thickness of less than 8.0 mm, a wet thickness of less than 12.5 mm, and a rewet less than or equal to 0.14 grams according to the Modified Fluid Intake Under Pressure Test as described herein.
Embodiment 13: The absorbent material of embodiment 12, wherein the intake layer and the absorbent layer provide an integrated material including an interface between the intake layer and the absorbent layer, the interface including at least some fibers of the intake layer with at least some fibers of the absorbent layer.
Embodiment 14: The absorbent material of embodiment 12 or 13, wherein the absorbent material further comprises a dry thickness of less than 7.0 mm.
Embodiment 15: The absorbent material of any one of embodiments 12-14, further comprising a saturation capacity greater than 120 grams according to the Modified Fluid Intake Under Pressure Test as described herein.
Embodiment 16: The absorbent material of any one of embodiments 12-15, wherein the intake layer comprises synthetic fibers, binder fibers, and a basis weight of between 20-120 gsm.
Embodiment 17: The absorbent material of any one of embodiments 12-16, wherein absorbent layer comprises absorbent fibers, binder fibers, and superabsorbent material.
Embodiment 18: An absorbent material comprising: an intake layer; and an absorbent layer; wherein the absorbent material comprises a saturation capacity greater than 125 grams, and a rewet of less than or equal to 0.14 grams and a wet thickness of less than 17 mm according to the Modified Fluid Intake Under Pressure Test as described herein.
Embodiment 19: The absorbent material of embodiment 18, wherein the intake layer and the absorbent layer provide an integrated material including an interface between the intake layer and the absorbent layer, the interface including at least some fibers of the intake layer with at least some fibers of the absorbent layer.
Embodiment 20: The absorbent material of embodiment 18 or 19, further comprising a second intake time of less than 50 seconds.
Embodiment 21: The absorbent material of any one of embodiments 18-20, further comprising a third intake time of less than 85 seconds.
Embodiment 22: The absorbent material of any one of embodiments 18-21, wherein the wet thickness is less than 12.5 mm.
Embodiment 23: The absorbent material of any one of embodiments 18-22, further comprising a dry thickness of less than 8.0 mm.
Embodiment 24: An absorbent material comprising: an intake layer, the intake layer comprising synthetic fibers and binder fibers, the intake layer comprising a basis weight less than 50 gsm; and an absorbent layer, the absorbent layer comprising superabsorbent material, cellulosic fibers, and binder fibers, wherein the binder fibers provide less than 20% of the absorbent layer (by total weight of the absorbent layer); wherein the intake layer and the absorbent layer provide an integrated material including an interface between the intake layer and the absorbent layer, the interface including at least some fibers of the intake layer with at least some fibers of the absorbent layer.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent material comprising:
an intake layer; and
an absorbent layer;
wherein the absorbent material comprises a saturation capacity greater than 125 grams, and a second intake time of less than 50 seconds and a wet thickness of less than 17 mm according to the Modified Fluid Intake Under Pressure Test as described herein.

2. The absorbent material of claim 1, wherein the intake layer and the absorbent layer provide an integrated material including an interface between the intake layer and the absorbent layer, the interface including at least some fibers of the intake layer mixed with at least some fibers of the absorbent layer.

3. The absorbent material of claim 1, wherein the saturation capacity is greater than 150 grams.

4. The absorbent material of claim 1, further comprising a third intake time of less than 85 seconds.

5. The absorbent material of claim 1, wherein the wet thickness is less than or equal to 14.0 mm.

6. The absorbent material of claim 1, further comprising a dry thickness less than 8.0 mm.

7. The absorbent material of claim 1, further comprising a rewet less than or equal to 0.14 grams.

8. The absorbent material of claim 1, wherein the intake layer comprises synthetic fibers and binder fibers.

9. The absorbent material of claim 8, wherein the binder fibers comprise between 15% to 50% of the intake layer (by total weight of the intake layer).

10. The absorbent material of claim 1, wherein the absorbent layer comprises absorbent fibers, binder fibers, and superabsorbent material, and wherein the binder fibers comprise less than 30% of the absorbent layer (by total weight of the intake layer).

11. The absorbent material of claim 1, wherein the intake layer comprises synthetic fibers and binder fibers, the binder fibers of the intake layer comprise less than 50% of the intake layer (by total weight of the intake layer); and wherein the absorbent layer comprises absorbent fibers, binder fibers, and superabsorbent material, wherein the binder fibers of the absorbent layer comprise less than 20% of the absorbent layer (by total weight of the absorbent layer).

12. An absorbent material comprising:
an intake layer; and
an absorbent layer;
wherein the absorbent material comprises a dry thickness of less than 8.0 mm, a wet thickness of less than 12.5 mm, and a rewet less than or equal to 0.14 grams according to the Modified Fluid Intake Under Pressure Test as described herein.

13. The absorbent material of claim 12, wherein the intake layer and the absorbent layer provide an integrated material including an interface between the intake layer and the absorbent layer, the interface including at least some fibers of the intake layer with at least some fibers of the absorbent layer.

14. The absorbent material of claim 12, wherein the absorbent material further comprises a dry thickness of less than 7.0 mm.

15. The absorbent material of claim 12, further comprising a saturation capacity greater than 120 grams according to the Modified Fluid Intake Under Pressure Test as described herein.

16. The absorbent material of claim 12, wherein the intake layer comprises synthetic fibers, binder fibers, and a basis weight of between 20-120 gsm.

17. The absorbent material of claim 12, wherein absorbent layer comprises absorbent fibers, binder fibers, and superabsorbent material.

18. An absorbent material comprising:
an intake layer; and
an absorbent layer;
wherein the absorbent material comprises a saturation capacity greater than 125 grams, and a rewet of less than or equal to 0.14 grams and a wet thickness of less than 17 mm according to the Modified Fluid Intake Under Pressure Test as described herein.

19. The absorbent material of claim 18, wherein the intake layer and the absorbent layer provide an integrated material including an interface between the intake layer and the absorbent layer, the interface including at least some fibers of the intake layer with at least some fibers of the absorbent layer.

20. The absorbent material of claim 18, further comprising a second intake time of less than 50 seconds.

21. The absorbent material of claim 18, further comprising a third intake time of less than 85 seconds.

22. The absorbent material of claim 18, wherein the wet thickness is less than 12.5 mm.

23. The absorbent material of claim 18, further comprising a dry thickness of less than 8.0 mm.

24. An absorbent material comprising:
an intake layer, the intake layer comprising synthetic fibers and binder fibers, the intake layer comprising a basis weight less than 50 gsm; and
an absorbent layer, the absorbent layer comprising superabsorbent material, cellulosic fibers, and binder fibers, wherein the binder fibers provide less than 20% of the absorbent layer (by total weight of the absorbent layer);
wherein the intake layer and the absorbent layer provide an integrated material including an interface between the intake layer and the absorbent layer, the interface including at least some fibers of the intake layer with at least some fibers of the absorbent layer.
